(19) United States
(12) Patent Application Publication
(10) Pub. No.: US 2025/0019449 A1
(43) Pub. Date: Jan. 16, 2025

(54) ANTI-CCR8 AGENTS

(71) Applicant: Memorial Sloan Kettering Cancer Center, New York, NY (US)

(72) Inventors: Alexander Y. Rudensky, New York, NY (US); George Plitas, New York, NY (US); Laura M. Walker, Lebanon, NH (US); Noel Pauli, Lebanon, NH (US); Robert Pejchal, Lebanon, NH (US); Cory Ahonen, Lebanon, NH (US)

(21) Appl. No.: 17/909,310

(22) PCT Filed: Mar. 5, 2021
(86) PCT No.: PCT/US21/21008

§ 371 Date: Sep. 2, 2022

Related U.S. Application Data
(60) Provisional application No. 62/985,827, filed on Mar. 5, 2020.

(51) Int. Cl. C07K 16/28; A61P 35/00

(52) U.S. Cl. CPC C07K 16/2866; A61P 35/00; A61K 2039/505; C07K 2317/20; C07K 2317/565; C07K 2317/77

(57) ABSTRACT

The present invention provides, among other things, methods and compositions for diagnosing and/or treating cancer by targeting CCR8. In particular, the present invention provides technologies for depleting Treg cells, and particularly tumor-infiltrating Treg cells.

### BACKGROUND

Significant effort has been invested in the identification and/or development for promoting the ability of the immune system to target and destroy tumors. Unfortunately, so far, success has proven elusive. Indeed, although therapeutic modulation of the immune system in cancer patients through, for example, antibody blockage of inhibitory molecules, adoptive T cell transfer, vaccination and other methods has shown some clinical benefit, patient responses have been variable at best.

### SUMMARY

The present invention recognizes a need for improved cancer treatments. The present disclosure recognizes that depletion of tumor infiltrating regulatory T cells (Tregs) improves immune response to tumors. The present disclosure recognizes that inadvertent targeting of other immune cells might inhibit, rather than promote, immune response to tumors.

Among other things, the present disclosure provides agents for the specific depletion of tumor infiltrating Tregs. For example, present disclosure provides particular anti-CCR8 agents (e.g., anti-CCR8 antibody agents), as well as technologies for making and/or using such agents and/or compositions that comprise and/or deliver them, and technologies for characterizing useful anti-CCR8 agents, including, for example, with reference to agents specifically exemplified herein.

In some embodiments, the present disclosure provides technologies for the depletion of tumor infiltrating Tregs.

In some embodiments the present disclosure provides compositions that are, comprise, and/or deliver an anti-CCR8 agent. In some embodiments, the present disclosure provides methods of treating cancer by targeting CCR8 in a subject having a tumor, so that tumor-infiltrating Treg cells are depleted in the subject. In some such embodiments, targeting CCR8 comprises administering to the subject a composition that comprises and/or delivers an anti-CCR8 agent as described herein. In some embodiments, administration of such a composition achieves depletion of tumor-infiltrating Treg cells.

In some embodiments, an anti-CCR8 agent provided herein binds specifically to CCR8; in some such embodiments, such an anti-CCR8 agent binds specifically to CCR8 in or on tumor-infiltrating Treg cells. In some embodiments, an anti-CCR8 agent is useful to bind CCR8 (e.g., in or on tumor-infiltrating Treg cells) and/or to block binding of an alternative binding partner to CCR8 (e.g., in or on such tumor-infiltrating Treg cells).

In some particular embodiments, a provided anti-CCR8 agent is or comprises an antibody agent.

In some embodiments, anti-CCR8 therapy as described herein is administered to a particular patient population; for example, in some embodiments a subject is suffering from or susceptible to development of one or more tumors determined or suspected to have been infiltrated by and/or to be at risk of infiltration by, Tregs.

In some embodiments, a subject may have received and/or be receiving therapy other than anti-CCR8 therapy as described herein; in some such embodiments, such other therapy might be or comprise other immune system promoting therapy (e.g., therapy that activates and/or supports a tumor-targeting immune response. Alternatively or additionally, in some embodiments, such other therapy might be or comprise chemotherapeutic therapy (e.g., designed to preferentially kill tumor cells) and/or pain therapy or other therapy that alleviates or avoids one or more symptoms or characteristics of cancer or of therapy received by the subject.

In some embodiments, a subject may be suffering from or susceptible to a cancer characterized by solid tumor(s).

In some embodiments, a provided anti-CCR8 agent may be used to detect and/or to quantify CCR8 present in a sample (e.g., a biological and/or environmental sample).

### BRIEF DESCRIPTION OF THE DRAWING

FIGS. 1A and 1B demonstrate binding of discovered antibodies to human CCR8. ADI-26140: Human IgG1 isotype control; CTL-33364: Biolegend CCR8 control IgG (L263G8).

FIGS. 2A and 2B demonstrate ADCC activity of discovered antibodies. FIG. 2A demonstrates induction of ADCC activity when the discovered antibodies interact with a high affinity FcγRIIIa. FIG. 2B demonstrates induction of ADCC activity when the discovered antibodies interact with a high affinity FcγRIIIa.

FIG. 3 demonstrates the ability of discovered antibodies to bind to tumor infiltrating regulatory T-cells. FACS histograms show the fluorescence intensity of a human IgG1 negative control (26140); a commercially available anti-CCR8 antibody (Biolgend; L263G8); and discovered antibodies. The ratio of the mean fluorescence intensity (MFI) of the discovered antibodies and the MFI of the L263G8 anti-CCR8 antibody is provided.

FIG. 4 demonstrates the ability of discovered antibodies to be internalized in vitro. Internalization of Zenon pHrodo iFLlabeled anti-CCR8 antibodies was measured by flow cytometry. Specific mean fluorescence intensity (MFI) was plotted as a function of antibody concentration (log μg/mL) and EC50 for internalization was determined. Anti-KTI and pHrodo only were utilized as negative controls.

### BRIEF DESCRIPTION OF THE SEQUENCES LISTING

SEQ ID Nos. 38-43 are exemplary heavy chain variable domains. SEQ ID Nos. 1-5 are exemplary heavy chain variable sequences described herein as CDR3 sequences. SEQ ID Nos. 6-9 are exemplary heavy chain variable sequences described herein as CDR1 sequences. SEQ ID Nos. 10-14 are exemplary heavy chain variable sequences described herein as CDR2 sequences. SEQ ID Nos. 50-54 are exemplary heavy chain variable sequences described herein as FR1 sequences. SEQ ID Nos. 55-59 are exemplary heavy chain variable sequences described herein as FR2 sequences. SEQ ID Nos. 55-58 are exemplary heavy chain variable sequences described herein as FR3 sequences. SEQ ID Nos. 59-61 are exemplary heavy chain variable sequences described herein as FR4 sequences. SEQ ID Nos. 26-31 are exemplary heavy chain variable domain nucleic acid sequences.

SEQ ID Nos. 44-49 are exemplary light chain variable domains. SEQ ID Nos. 15-18 are exemplary light chain variable sequences described herein as CDR1 sequences. SEQ ID Nos. 19-21 are exemplary light chain variable sequences described herein as CDR2 sequences. SEQ ID Nos. 22-25 are exemplary light chain variable sequences described herein as CDR3 sequences. SEQ ID Nos. 62-64 are exemplary light chain variable sequences described herein as FR1 sequences. SEQ ID Nos. 68-71 are exemplary light chain variable sequences described herein as FR2 sequences. SEQ ID Nos. 74-76 are exemplary light chain variable sequences described herein as FR3 sequences. SEQ ID Nos. 80-81 are exemplary light chain variable sequences described herein as FR4 sequences. SEQ ID Nos. 32-37 are exemplary light chain variable domain nucleic acid sequences.

### Definitions

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Activating agent: As used herein, the term “activating agent” refers to an agent whose presence or level correlates with elevated level or activity of a target, as compared with that observed absent the agent (or with the agent at a different level). In some embodiments, an activating agent is one whose presence or level correlates with a target level or activity that is comparable to or greater than a particular reference level or activity (e.g., that observed under appropriate reference conditions, such as presence of a known activating agent, e.g., a positive control).

Administration: As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc., In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Affinity: As is known in the art, “affinity” is a measure of the tightness with a particular ligand binds to its partner. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

Affinity matured” (or “affinity matured antibody”): as used herein, refers to an antibody with one or more alterations in one or more CDRs thereof which result an improvement in the affinity of the antibody for antigen, compared to a parent antibody which does not possess those alteration(s). In some embodiments, affinity matured antibodies will have nanomolar or even picomolar affinities for a target antigen. Affinity matured antibodies may be produced by any of a variety of procedures known in the art. Marks et al., BioTechnology 10:779-783 (1992) describes affinity maturation by V_{H} and V_{L} domain shuffling. Random mutagenesis of CDR and/or framework residues is described by: Barbas et al. Proc. Nat. Acad. Sci. U.S.A. 91:3809-3813 (1994); Schier et al., Gene 169:147-155 (1995); Yelton et al., J. Immunol. 155:1994-2004 (1995); Jackson et al., J. Immunol. 154(7):3310-9 (1995); and Hawkins et al., J. Mol. Biol. 226:889-896 (1992).

Agent: In general, the term “agent”, as used herein, may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Agonist: Those skilled in the art will appreciate that the term “agonist” may be used to refer to an agent condition, or event whose presence, level, degree, type, or form correlates with increased level or activity of another agent (i.e., the agonized target). In general, an agonist may be or include an agent of any chemical class including, for example, small molecules, polypeptides, nucleic acids, carbohydrates, lipids, metals, and/or any other entity that shows the relevant activating activity. In some embodiments, an agonist may be direct (in which case it exerts its influence directly upon its target); in some embodiments, an agonist may be indirect (in which case it exerts its influence by other than binding to its target; e.g., by interacting with a regulator of the target, so that level or activity of the target is altered).

Amino acid: in its broadest sense, as used herein, refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid: in some embodiments, an amino acid is a D-amino acid: in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Antagonist: Those skilled in the art will appreciate that the term “antagonist”, as used herein, may be used to refer to an agent condition, or event whose presence, level, degree, type, or form correlates with decreased level or activity of another agent (i.e., the inhibited agent, or target). In general, an antagonist may be or include an agent of any chemical class including, for example, small molecules, polypeptides, nucleic acids, carbohydrates, lipids, metals, and/or any other entity that shows the relevant inhibitory activity. In some embodiments, an antagonist may be direct (in which case it exerts its influence directly upon its target); in some embodiments, an antagonist may be indirect (in which case it exerts its influence by other than binding to its target; e.g., by interacting with a regulator of the target, so that level or activity of the target is altered).

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal: in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE® s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR® s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.].

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc, as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with the present invention is in a format selected from, but not limited to, intact IgA, IgG, IgE or IgM antibodies; bi- or multi-specific antibodies (e.g., Zybodies®), etc); antibody fragments such as Fab fragments, Fab′ fragments, F(ab)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE® s; ankyrin repeat proteins or DARPINS®; Avimers®; DARTs; TCR-like antibodies; Adnectins®; Affilins®; Trans-bodies®; Affibodies®; TrimerX®; MicroProteins; Fynomers®, Centyrins®; and KALBITOR® s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR): in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Antibody-Dependent Cellular Cytotoxicity: As used herein, the term “antibody-dependent cellular cytotoxicity” or “ADCC” refers to a phenomenon in which target cells bound by antibody are killed by immune effector cells. Without wishing to be bound by any particular theory, we observe that ADCC is typically understood to involve Fc receptor (FcR)-bearing effector cells can recognizing and subsequently killing antibody-coated target cells (e.g., cells that express on their surface specific antigens to which an antibody is bound). Effector cells that mediate ADCC can include immune cells, including but not limited to one or more of natural killer (NK) cells, macrophage, neutrophils, eosinophils.

Antibody fragment: As used herein, an “antibody fragment” refers to a portion of an antibody or antibody agent as described herein, and typically refers to a portion that includes an antigen-binding portion or variable region thereof. An antibody fragment may be produced by any means. For example, in some embodiments, an antibody fragment may be enzymatically or chemically produced by fragmentation of an intact antibody or antibody agent. Alternatively, in some embodiments, an antibody fragment may be recombinantly produced (i.e., by expression of an engineered nucleic acid sequence. In some embodiments, an antibody fragment may be wholly or partially synthetically produced. In some embodiments, an antibody fragment (particularly an antigen-binding antibody fragment) may have a length of at least about 50, 60, 70, 80, 90, 100, 110, 120, 130, 140, 150, 160, 170, 180, 190 amino acids or more, in some embodiments at least about 200 amino acids.

Antigen: The term “antigen”, as used herein, refers to an agent that elicits an immune response; and/or (ii) an agent that binds to a T cell receptor (e.g., when presented by an MHC molecule) or to an antibody. In some embodiments, an antigen elicits a humoral response (e.g., including production of antigen-specific antibodies); in some embodiments, an elicits a cellular response (e.g., involving T-cells whose receptors specifically interact with the antigen). In some embodiments, and antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer [e.g., other than a nucleic acid or amino acid polymer) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen.

Antigen presenting cell: The phrase “antigen presenting cell” or “APC,” as used herein, has its art understood meaning referring to cells which process and present antigens to T-cells. Exemplary antigen cells include dendritic cells, macrophages and certain activated epithelial cells.

Associated: Two events or entities are “associated” with one another, as that term is used herein, if the presence, level and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Binding domain: As used herein, refers to a moiety or entity that specifically binds to a target moiety or entity. Typically, the interaction between a binding domain and its target is non-covalent. In some embodiments, a binding domain may be or comprise a moiety or entity of any chemical class including, for example, a carbohydrate, a lipid, a nucleic acid, a metal, a polypeptide, a small molecule. In some embodiments, a binding domain may be or comprise a polypeptide (or complex thereof). In some embodiments, a binding domain may be or comprise a target-binding portion of an antibody agent, a cytokine, a ligand (e.g., a receptor ligand), a receptor, a toxin, etc. In some embodiments, a binding domain may be or comprise an aptamer. In some embodiments, a binding domain may be or comprise a peptide nucleic acid (PNA).

Binding: It will be understood that the term “binding”, as used herein, typically refers to a non-covalent association between or among two or more entities. “Direct” binding involves physical contact between entities or moieties: indirect binding involves physical interaction by way of physical contact with one or more intermediate entities. Binding between two or more entities can typically be assessed in any of a variety of contexts-including where interacting entities or moieties are studied in isolation or in the context of more complex systems (e.g., while covalently or otherwise associated with a carrier entity and/or in a biological system or cell).

Biological Sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Biomarker: The term “biomarker” is used herein, consistent with its use in the art, to refer to a to an entity whose presence, level, or form, correlates with a particular biological event or state of interest, so that it is considered to be a “marker” of that event or state. To give but a few examples, in some embodiments, a biomarker may be or comprise a marker for a particular disease state, or for likelihood that a particular disease, disorder or condition may develop, occur, or reoccur. In some embodiments, a biomarker may be or comprise a marker for a particular disease or therapeutic outcome, or likelihood thereof. Thus, in some embodiments, a biomarker is predictive, in some embodiments, a biomarker is prognostic, in some embodiments, a biomarker is diagnostic, of the relevant biological event or state of interest. A biomarker may be an entity of any chemical class. For example, in some embodiments, a biomarker may be or comprise a nucleic acid, a polypeptide, a lipid, a carbohydrate, a small molecule, an inorganic agent (e.g., a metal or ion), or a combination thereof. In some embodiments, a biomarker is a cell surface marker. In some embodiments, a biomarker is intracellular. In some embodiments, a biomarker is found outside of cells (e.g., is secreted or is otherwise generated or present outside of cells, e.g., in a body fluid such as blood, urine, tears, saliva, cerebrospinal fluid, etc.

Bispecific antibody: as used herein, refers to a bispecific binding agent in which at least one, and typically both, of the binding moieties is or comprises an antibody component. A variety of different bi-specific antibody structures are known in the art. In some embodiments, each binding moiety in a bispecific antibody that is or comprises an antibody component includes V_{H} and/or V_{L} regions; in some such embodiments, the V_{H} and/or V_{L} regions are those found in a particular monoclonal antibody. In some embodiments, where the bispecific antibody contains two antibody component-binding moieties, each includes V_{H} and/or V_{L} regions from different monoclonal antibodies. In some embodiments, where the bispecific antibody contains two antibody component binding moieties, wherein one of the two antibody component binding moieties includes an immunoglobulin molecule having V_{H} and/or V_{L} regions that contain CDRs from a first monoclonal antibody, and one of the two antibody component binding moieties includes an antibody fragment (e.g., Fab, F(ab′), F(ab′)₂, Fd, Fv, dAB, scFv, etc.) having V_{H} and/or V_{L} regions that contain CDRs from a second monoclonal antibody.

Bispecific binding agent: as used herein, refers to a polypeptide agent with two discrete binding moieties, each of which binds with a distinct target. In some embodiments, a bispecific binding agent is or comprises a single polypeptide; in some embodiments, a bispecific binding agent is or comprises a plurality of peptides which, in some such embodiments may be covalently associated with one another, for example by cross-linking. In some embodiments, the two binding moieties of a bispecific binding agent recognize different sites (e.g., epitopes) the same target (e.g., antigen); in some embodiments, they recognize different targets. In some embodiments, a bispecific binding agent is capable of binding simultaneously to two targets that are of different structure.

Cancer: The terms “cancer”, “malignancy”, “neoplasm”, “tumor”, and “carcinoma”, are used herein to refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor.

CDR: as used herein, refers to a complementarity determining region within an antibody variable region. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. Certain systems have been established in the art for defining CDR boundaries (e.g., Kabat, Chothia, etc.); those skilled in the art appreciate the differences between and among these systems and are capable of understanding CDR boundaries to the extent required to understand and to practice the claimed invention.

Cellular lysate: As used herein, the term “cellular lysate” or “cell lysate” refers to a fluid containing contents of one or more disrupted cells (i.e., cells whose membrane has been disrupted). In some embodiments, a cellular lysate includes both hydrophilic and hydrophobic cellular components. In some embodiments, a cellular lysate includes predominantly hydrophilic components; in some embodiments, a cellular lysate includes predominantly hydrophobic components. In some embodiments, a cellular lysate is a lysate of one or more cells selected from the group consisting of plant cells, microbial (e.g., bacterial or fungal) cells, animal cells (e.g., mammalian cells), human cells, and combinations thereof. In some embodiments, a cellular lysate is a lysate of one or more abnormal cells, such as cancer cells. In some embodiments, a cellular lysate is a crude lysate in that little or no purification is performed after disruption of the cells; in some embodiments, such a lysate is referred to as a “primary” lysate. In some embodiments, one or more isolation or purification steps is performed on a primary lysate; however, the term “lysate” refers to a preparation that includes multiple cellular components and not to pure preparations of any individual component.

Chemotherapeutic Agent: The term “chemotherapeutic agent”, has used herein has its art-understood meaning referring to one or more pro-apoptotic, cytostatic and/or cytotoxic agents, for example specifically including agents utilized and/or recommended for use in treating one or more diseases, disorders or conditions associated with undesirable cell proliferation. In many embodiments, chemotherapeutic agents are useful in the treatment of cancer. In some embodiments, a chemotherapeutic agent may be or comprise one or more alkylating agents, one or more anthracyclines, one or more cytoskeletal disruptors (e.g. microtubule targeting agents such as taxanes, maytansine and analogs thereof, of), one or more epothilones, one or more histone deacetylase inhibitors HDACs), one or more topoisomerase inhibitors (e.g., inhibitors of topoisomerase I and/or topoisomerase II), one or more kinase inhibitors, one or more nucleotide analogs or nucleotide precursor analogs, one or more peptide antibiotics, one or more platinum-based agents, one or more retinoids, one or more vinca alkaloids, and/or one or more analogs of one or more of the following (i.e., that share a relevant anti-proliferative activity). In some particular embodiments, a chemotherapeutic agent may be or comprise one or more of Actinomycin, All-trans retinoic acid, an Auiristatin, Azacitidine, Azathioprine, Bleomycin, Bortezomib, Carboplatin, Capecitabine, Cisplatin, Chlorambucil, Cyclophosphamide, Curcumin, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Maytansine and/or analogs thereof (e.g. DM1) Mechlorethamine, Mercaptopurine, Methotrexate, Mitoxantrone, a Maytansinoid, Oxaliplatin, Paclitaxel, Pemetrexed, Teniposide, Tioguanine, Topotecan, Valrubicin, Vinblastine, Vincristine, Vindesine, Vinorelbine, and combinations thereof. In some embodiments, a chemotherapeutic agent may be utilized in the context of an antibody-drug conjugate. In some embodiments, a chemotherapeutic agent is one found in an antibody-drug conjugate selected from the group consisting of: hLL1-doxorubicin, hRS7-SN-38, hMN-14-SN-38, hLL2-SN-38, hA20-SN-38, hPAM4-SN-38, hLL1-SN-38, hRS7-Pro-2-P-Dox, hMN-14-Pro-2-P-Dox, hLL2-Pro-2-P-Dox, hA20)-Pro-2-P-Dox, hPAM4-Pro-2-P-Dox, hLL1-Pro-2-P-Dox, P4/D10-doxorubicin, gemtuzumab ozogamicin, brentuximab vedotin, trastuzumab emtansine, inotuzumab ozogamicin, glembatumomab vedotin, SAR3419, SAR566658, BIIB015, BT062, SGN-75, SGN-CD19A, AMG-172, AMG-595, BAY-94-9343, ASG-5ME, ASG-22ME, ASG-16M8F, MDX-1203, MLN-0264, anti-PSMA ADC, RG-7450, RG-7458, RG-7593, RG-7596, RG-7598, RG-7599, RG-7600, RG-7636, ABT-414, IMGN-853, IMGN-529, vorsetuzumab mafodotin, and lorvotuzumab mertansine. In some embodiments, a chemotherapeutic agent may be one described as utilized in an antibody-drug conjugate as described or discussed in one or more of Govindan et al, TheScientificWorld JOURNAL 2010, 10:2070-2089. In some embodiments, a chemotherapeutic agent may be or comprise one or more of farnesyl-thiosalicylic acid (FTS), 4-(4-Chloro-2-methylphenoxy)-N-hydroxy butanamide (CMH), estradiol (E2), tetramethoxystilbene (TMS), δ-tocatrienol, salinomycin, or curcumin.

Combination Therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality (ies) to a subject receiving the other agent(s) or modality (ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity). In some embodiments, two or more agents may be administered simultaneously; in some embodiments, such agents may be administered sequentially; in some embodiments, such agents are administered in overlapping dosing regimens.

Chimeric antibody: as used herein, refers to an antibody whose amino acid sequence includes V_{H} and V_{L} region sequences that are found in a first species and constant region sequences that are found in a second species, different from the first species. In many embodiments, a chimeric antibody has murine V_{H} and V_{L} regions linked to human constant regions. In some embodiments, an antibody with human V_{H} and V_{L} regions linked to non-human constant regions (e.g., a mouse constant region) is referred to as a “reverse chimeric antibody”.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition”, as used herein, may be used to refer to a discrete physical entity that comprises one or more specified components. In general, unless otherwise specified, a composition may be of any form—e.g., gas, gel, liquid, solid, etc.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Derivative: As used herein, the term “derivative” refers to a structural analogue of a reference substance. That is, a “derivative” is a substance that shows significant structural similarity with the reference substance, for example sharing a core or consensus structure, but also differs in certain discrete ways. In some embodiments, a derivative is a substance that can be generated from the reference substance by chemical manipulation. In some embodiments, a derivative is a substance that can be generated through performance of a synthetic process substantially similar to (e.g., sharing a plurality of steps with) one that generates the reference substance.

Detectable entity: The term “detectable entity” as used herein refers to any element, molecule, functional group, compound, fragment or moiety that is detectable. In some embodiments, a detectable entity is provided or utilized alone. In some embodiments, a detectable entity is provided and/or utilized in association with (e.g., joined to) another agent. Examples of detectable entities include, but are not limited to: various ligands, radionuclides (e.g., ³H, ¹⁴C, ¹⁸F. ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available.

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide; in some such embodiments, a domain is characterized by a particular structural element (e.g., a particular amino acid sequence or sequence motif, α-helix character, β-sheet character, coiled-coil character, random coil character, etc.), and/or by a particular functional feature (e.g., binding activity, enzymatic activity, folding activity, signaling activity, etc.).

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Epitope: as used herein, includes any moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Excipient: as used herein, refers to an inactive (e.g., non-therapeutic) agent that may be included in a pharmaceutical composition, for example to provide or contribute to a desired consistency or stabilizing effect. In some embodiments, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Fc ligand: as used herein refers to a molecule, preferably a polypeptide, from any organism that binds to the Fc region of an antibody to form an Fc-ligand complex. Fc ligands include but are not limited to FcγRIIA (CD32A), FcγRIIB (CD32B), FcγRIIIA (CD16A), FcγRIIIB (CD16B), FcγRI (CD64), FcεRII (CD23), FcRn, Clq, C3, staphylococcal protein A, streptococcal protein G, and viral FcγR. Fc ligands may include undiscovered molecules that bind Fc.

Framework” or “framework region”: as used herein, refers to the sequences of a variable region minus the CDRs. Because a CDR sequence can be determined by different systems, likewise a framework sequence is subject to correspondingly different interpretations. The six CDRs divide the framework regions on the heavy and light chains into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, FR1, for example, represents the first framework region closest to the amino terminal end of the variable region and 5′ with respect to CDR1, and FRs represents two or more of the sub-regions constituting a framework region.

Functional: As used herein, a “functional” biological molecule is a biological molecule in a form in which it exhibits a property and/or activity by which it is characterized. A biological molecule may have two functions (i.e., bifunctional) or many functions (i.e., multifunctional).

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the whole.

Gene: As used herein, the term “gene” refers to a DNA sequence in a chromosome that codes for a product (e.g., an RNA product and/or a polypeptide product). In some embodiments, a gene includes coding sequence (i.e., sequence that encodes a particular product): in some embodiments, a gene includes non-coding sequence. In some particular embodiments, a gene may include both coding (e.g., exonic) and non-coding (e.g., intronic) sequences. In some embodiments, a gene may include one or more regulatory elements that, for example, may control or impact one or more aspects of gene expression (e.g., cell-type-specific expression, inducible expression, etc.).

Gene product or expression product. As used herein, the term “gene product” or “expression product” generally refers to an RNA transcribed from the gene (pre- and/or post-processing) or a polypeptide (pre- and/or post-modification) encoded by an RNA transcribed from the gene.

Genome: As used herein, the term “genome” refers to the total genetic information carried by an individual organism or cell, represented by the complete DNA sequences of its chromosomes.

High affinity binding: The term “high affinity binding”, as used herein refers to a high degree of tightness with which a particular ligand binds to its partner. Affinities can be measured by any available method, including those known in the art. In some embodiments, binding is considered to be high affinity if the K_{d} is about 500 pM or less (e.g., below about 400 pM, about 300 pM, about 200 pM, about 100 pM, about 90 pM, about 80 pM, about 70 pM, about 60 pM, about 50 pM, about 40 pM, about 30 pM, about 20 pM, about 10 pM, about 5 pM, about 4 pM, about 3 pM, about 2 pM, etc.) in binding assays. In some embodiments, binding is considered to be high affinity if the affinity is stronger (e.g., the K_{d} is lower) for a polypeptide of interest than for a selected reference polypeptide. In some embodiments, binding is considered to be high affinity if the ratio of the K_{d} for a polypeptide of interest to the K_{d} for a selected reference polypeptide is 1:1 or less (e.g., 0.9:1, 0.8:1, 0.7:1, 0.6:1, 0.5:1. 0.4:1, 0.3:1, 0.2:1, 0.1:1, 0.05:1, 0.01:1, or less). In some embodiments, binding is considered to be high affinity if the K_{d} for a polypeptide of interest is about 100% or less (e.g., about 99%, about 98%, about 97%, about 96%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5%, about 4%, about 3%, about 2%, about 1% or less) of the K_{d} for a selected reference polypeptide.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution. Typical amino acid categorizations are summarized below:

| | | | | | |
|---|---|---|---|---|---|
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position: when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences. Representative algorithms and computer programs useful in determining the percent homology between two nucleotide sequences include, for example, the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent homology between two nucleotide sequences can, alternatively, be determined for example using the GAP program in the GCG software package using an NWSgapdna.CMP matrix.

Host cell: as used herein, refers to a cell into which exogenous DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous DNA (e.g., a recombinant nucleic acid sequence). Exemplary cells include those of prokaryotes and eukaryotes (single-cell or multiple-cell), bacterial cells (e.g., strains of E. coli. Bacillus spp., Streptomyces spp., etc.), mycobacteria cells, fungal cells, yeast cells (e.g., S. cerevisiae. S. pombe. P. pastoris. P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: CHO (e.g., CHO K1, DXB-1 1 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3 A cell, HT1080 cell, myeloma cell, tumor cell, and a cell line derived from an aforementioned cell. In some embodiments, the cell comprises one or more viral genes.

Human: In some embodiments, a human is an embryo, a fetus, an infant, a child, a teenager, an adult, or a senior citizen.

Human antibody: as used herein, is intended to include antibodies having variable and constant regions generated (or assembled) from human immunoglobulin sequences. In some embodiments, antibodies (or antibody components) may be considered to be “human” even though their amino acid sequences include residues or elements not encoded by human germline immunoglobulin sequences (e.g., include sequence variations, for example that may (originally) have been introduced by random or site-specific mutagenesis in vitro or by somatic mutation in vivo), for example in one or more CDRs and in particular CDR3.

Humanized: as is known in the art, the term “humanized” is commonly used to refer to antibodies (or antibody components) whose amino acid sequence includes V_{H} and V_{L} region sequences from a reference antibody raised in a non-human species (e.g., a mouse), but also includes modifications in those sequences relative to the reference antibody intended to render them more “human-like”, i.e., more similar to human germline variable sequences. In some embodiments, a “humanized” antibody (or antibody component) is one that immunospecifically binds to an antigen of interest and that has a framework (FR) region having substantially the amino acid sequence as that of a human antibody, and a complementary determining region (CDR) having substantially the amino acid sequence as that of a non-human antibody. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor immunoglobulin) and all or substantially all of the framework regions are those of a human immunoglobulin consensus sequence. In some embodiments, a humanized antibody also comprises at least a portion of an immunoglobulin constant region (Fc), typically that of a human immunoglobulin constant region. In some embodiments, a humanized antibody contains both the light chain as well as at least the variable domain of a heavy chain. The antibody also may include a C_{H}1, hinge, C_{H}2, C_{H}3, and, optionally, a C_{H}4 region of a heavy chain constant region. In some embodiments, a humanized antibody only contains a humanized V_{L} region. In some embodiments, a humanized antibody only contains a humanized V_{H} region. In some certain embodiments, a humanized antibody contains humanized V_{H} and V_{L} regions.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna. CMP matrix.

“Improve,” “increase”, “inhibit” or “reduce”: As used herein, the terms “improve”, “increase”, “inhibit”, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

In vitro: The term “in vitro” as used herein refers to events that occur in an artificial environment, e.g., in a test tube or reaction vessel, in cell culture, etc., rather than within a multi-cellular organism.

In vivo: as used herein refers to events that occur within a multi-cellular organism, such as a human and a non-human animal. In the context of cell-based systems, the term may be used to refer to events that occur within a living cell (as opposed to, for example, in vitro systems).

Isolated: as used herein, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

K_{D}: as used herein, refers to the dissociation constant of a binding agent (e.g., an antibody or binding component thereof) from a complex with its partner (e.g., the epitope to which the antibody or binding component thereof binds).

K_{off}: as used herein, refers to the off rate constant for dissociation of a binding agent (e.g., an antibody or binding component thereof) from a complex with its partner (e.g., the epitope to which the antibody or binding component thereof binds).

Kₒₙ: as used herein, refers to the on rate constant for association of a binding agent (e.g., an antibody or binding component thereof) with its partner (e.g., the epitope to which the antibody or binding component thereof binds).

Linker: as used herein, is used to refer to that portion of a multi-element agent that connects different elements to one another. For example, those of ordinary skill in the art appreciate that a polypeptide whose structure includes two or more functional or organizational domains often includes a stretch of amino acids between such domains that links them to one another. In some embodiments, a polypeptide comprising a linker element has an overall structure of the general form S1-L-S2, wherein S1 and S2 may be the same or different and represent two domains associated with one another by the linker. In some embodiments, a polyptide linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length. In some embodiments, a linker is characterized in that it tends not to adopt a rigid three-dimensional structure, but rather provides flexibility to the polypeptide. A variety of different linker elements that can appropriately be used when engineering polypeptides (e.g., fusion polypeptides) known in the art (see e.g., Holliger, P., et al. (1993) Proc. Natl. Acad. Sci. USA 90:6444-6448; Poljak, R. J., et al. (1994) Structure 2: 1 121-1123).

Low affinity binding: The term “low affinity binding”, as used herein refers to a low degree of tightness with which a particular ligand binds to its partner. As described herein, affinities can be measured by any available method, including methods known in the art. In some embodiments, binding is considered to be low affinity if the K_{d} is about 100 pM or more (e.g., above about 200 pM, 300 pM, 400 pM, 500 pM, 600 pM, 700 pM, 800 pM, 900 pM, 1 nM, 1.1 nM, 1.2 nM, 1.3 nM, 1.4 nM, 1.5 nM, etc.) In some embodiments, binding is considered to be low affinity if the affinity is the same or lower (e.g., the K_{d} is about the same or higher) for a polypeptide of interest than for a selected reference polypeptide. In some embodiments, binding is considered to be low affinity if the ratio of the K_{d} for a polypeptide of interest to the K_{d} for a selected reference polypeptide is 1:1 or more (e.g., 1.1:1, 1.2:1, 1.3:1, 1.4:1, 1.5:1. 1.6:1, 1.7:1, 1.8:1, 1.9:1, 2:1, 3:1, 4:1, 5:1, 10:1 or more). In some embodiments, binding is considered to be low affinity if the K_{d} for a polypeptide of interest is 100% or more (e.g., 100%, 105%, 110%, 115%, 120%, 125%, 130%, 135%, 140%, 145%, 150%, 155%, 160%, 165%, 170%, 175%, 180%, 185%, 190%, 195%, 200%, 300%, 400%, 500%, 1000%, or more) of the K_{d} for a selected reference polypeptide.

Mutant: As used herein, the term “mutant” refers to an entity that shows significant structural identity with a reference entity but differs structurally from the reference entity in the presence or level of one or more chemical moieties as compared with the reference entity. In many embodiments, a mutant also differs functionally from its reference entity. In general, whether a particular entity is properly considered to be a “mutant” of a reference entity is based on its degree of structural identity with the reference entity. As will be appreciated by those skilled in the art, any biological or chemical reference entity has certain characteristic structural elements. A mutant, by definition, is a distinct chemical entity that shares one or more such characteristic structural elements. To give but a few examples, a small molecule may have a characteristic core structural element (e.g., a macrocycle core) and/or one or more characteristic pendent moieties so that a mutant of the small molecule is one that shares the core structural element and the characteristic pendent moieties but differs in other pendent moieties and/or in types of bonds present (single vs double, E vs Z, etc.) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. For example, a mutant polypeptide may differ from a reference polypeptide as a result of one or more differences in amino acid sequence and/or one or more differences in chemical moieties (e.g., carbohydrates, lipids, etc.) covalently attached to the polypeptide backbone. In some embodiments, a mutant polypeptide shows an overall sequence identity with a reference polypeptide that is at least 85%, 86%, 87%, 88%, 89%, 90%, 91%, 92%, 93%, 94%, 95%, 96%, 97%, or 99%. Alternatively or additionally, in some embodiments, a mutant polypeptide does not share at least one characteristic sequence element with a reference polypeptide. In some embodiments, the reference polypeptide has one or more biological activities. In some embodiments, a mutant polypeptide shares one or more of the biological activities of the reference polypeptide. In some embodiments, a mutant polypeptide lacks one or more of the biological activities of the reference polypeptide. In some embodiments, a mutant polypeptide shows a reduced level of one or more biological activities as compared with the reference polypeptide.

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxy thymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0 (6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Operably linked: as used herein, refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with the coding elements of interest; in some embodiments, control elements act in trans to or otherwise at a from the functional element of interest.

Patient: As used herein, the term “patient” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is or includes cancer, or presence of one or more tumors. In some embodiments, the patient is receiving or has received certain therapy to diagnose and/or to treat a disease, disorder, or condition.

Peptide: The term “peptide” as used herein refers to a polypeptide that is typically relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids.

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the term “pharmaceutically acceptable” applied to the carrier, diluent, or excipient used to formulate a composition as disclosed herein means that the carrier, diluent, or excipient must be compatible with the other ingredients of the composition and not deleterious to the recipient thereof.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soy bean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Polypeptide: As used herein refers to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Recombinant: as used herein, is intended to refer to polypeptides that are designed, engineered, prepared, expressed, created, manufactured, and/or or isolated by recombinant means, such as polypeptides expressed using a recombinant expression vector transfected into a host cell, polypeptides isolated from a recombinant, combinatorial human polypeptide library (e.g., Hoogenboom, TIB Tech 15:62, 1997; Azzazy Clin. Biochem. 35:425, 2002; Gavilondo BioTechniques 29:128, 2002; Hoogenboom Immunology Today 21:371, 2000), antibodies isolated from an animal (e.g., a mouse) that is transgenic for human immunoglobulin genes (see e.g., Taylor Nuc. Acids Res. 20:6287, 1992; Little Immunology Today 12:364, 2000; Kellermann Curr. Opin. Biotechnol 13:593, 2002; Murphy Proc. Natl Acad Sci USA 111:5153, 2104) or polypeptides prepared, expressed, created or isolated by any other means that involves splicing selected sequence elements to one another. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source. For example, in some embodiments, a recombinant antibody polypeptide is comprised of sequences found in the germline of a source organism of interest (e.g., human, mouse, etc.). In some embodiments, a recombinant antibody has an amino acid sequence that resulted from mutagenesis (e.g., in vitro or in vivo, for example in a transgenic animal), so that the amino acid sequences of the V_{H} and V_{L} regions of the recombinant antibodies are sequences that, while originating from and related to germline V_{H} and V_{L} sequences, may not naturally exist within the germline antibody repertoire in vivo.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Response: As used herein, a response to treatment may refer to any beneficial alteration in a subject's condition that occurs as a result of or correlates with treatment. Such alteration may include stabilization of the condition (e.g., prevention of deterioration that would have taken place in the absence of the treatment), amelioration of symptoms of the condition, and/or improvement in the prospects for cure of the condition, etc. It may refer to a subject's response or to a tumor's response. Tumor or subject response may be measured according to a wide variety of criteria, including clinical criteria and objective criteria. Techniques for assessing response include, but are not limited to, clinical examination, positron emission tomatography, chest X-ray CT scan, MRI, ultrasound, endoscopy, laparoscopy, presence or level of tumor markers in a sample obtained from a subject, cytology, and/or histology. Many of these techniques attempt to determine the size of a tumor or otherwise determine the total tumor burden. Methods and guidelines for assessing response to treatment are discussed in Therasse et. al., “New guidelines to evaluate the response to treatment in solid tumors”, European Organization for Research and Treatment of Cancer, National Cancer Institute of the United States, National Cancer Institute of Canada, J. Natl. Cancer Inst., 2000, 92(3):205-216. The exact response criteria can be selected in any appropriate manner, provided that when comparing groups of tumors and/or patients, the groups to be compared are assessed based on the same or comparable criteria for determining response rate. One of ordinary skill in the art will be able to select appropriate criteria.

Sample: As used herein, the term “sample” typically refers to an aliquot of material obtained or derived from a source of interest. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow; blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secreations, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Solid Tumor: As used herein, the term “solid tumor” refers to an abnormal mass of tissue that usually does not contain cysts or liquid areas. In some embodiments, a solid tumor may be benign; in some embodiments, a solid tumor may be malignant. Those skilled in the art will appreciate that different types of solid tumors are typically named for the type of cells that form them. Examples of solid tumors are carcinomas, lymphomas, and sarcomas.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial identity: as used herein refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., Basic local alignment search tool, J. Mol. Biol., 215(3): 403-410, 1990; Altschul et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al, (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues. In the context of a CDR, reference to “substantial identity” typically refers to a CDR having an amino acid sequence at least 80%, preferably at least 85%, at least 90%, at least 95%, at least 98% or at least 99% identical to that of a reference CDR.

Substantial sequence homology: The phrase “substantial homology” is used herein to refer to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues will appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains Substitution of one amino acid for another of the same type may often be considered a “homologous” substitution. Typical amino acid categorizations are summarized below:

| | | | | | |
|---|---|---|---|---|---|
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology: Altschul, et al., “Gapped BLAST and PSI-BLAST: a new generation of protein database search programs”, Nucleic Acids Res. 25:3389-3402, 1997: Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology. In some embodiments, two sequences are considered to be substantially homologous if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are homologous over a relevant stretch of residues. In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues.

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Targets: As used herein, the term “targets” in general refers to specific binding under relevant conditions (e.g., in biological context and/or in presence of one or more particular competitors).

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutically Effective Amount: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. For example, in some embodiments, term “therapeutically effective amount”, refers to an amount which, when administered to an individual in need thereof in the context of inventive therapy, will block, stabilize, attenuate, or reverse a disease (e.g., cancer)-supportive process occurring in said individual, or will enhance or increase a disease (e.g., cancer)-suppressive process in said individual. In the context of cancer treatment, a “therapeutically effective amount” is an amount which, when administered to an individual diagnosed with a cancer, will prevent, stabilize, inhibit, or reduce the further development of such cancer in the individual. A particularly preferred “therapeutically effective amount” of a composition described herein reverses (in a therapeutic treatment) the development of a malignancy (e.g., a cancer such as a breast, colon, gastric, lung, and/or ovarian malignancy, etc) or helps achieve or prolong remission of a malignancy. A therapeutically effective amount administered to an individual to treat a cancer (e.g., a breast, colon, gastric, lung, and/or ovarian cancer) in that individual may be the same or different from a therapeutically effective amount administered to promote remission or inhibit metastasis. As with most cancer therapies, the therapeutic methods described herein are not to be interpreted as, restricted to, or otherwise limited to a “cure” for such cancer; rather the methods of treatment are directed to the use of the described compositions to “treat” a cancer (e.g., a breast, colon, gastric, lung, and/or ovarian cancer), i.e., to effect a desirable or beneficial change in the health of an individual who has such cancer. Such benefits are recognized by skilled healthcare providers in the field of oncology and include, but are not limited to, a stabilization of patient condition, a decrease in tumor size (tumor regression), an improvement in vital functions (e.g., improved function of cancerous tissues or organs), a decrease or inhibition of further metastasis, a decrease in opportunistic infections, an increased survivability, a decrease in pain, improved motor function, improved cognitive function, improved feeling of energy (vitality, decreased malaise), improved feeling of well-being, restoration of normal appetite, restoration of healthy weight gain, and combinations thereof. In addition, regression of a particular tumor in an individual (e.g., as the result of treatments described herein) may also be assessed by taking samples of cancer cells from the site of a tumor (e.g., a solid tumor such as a breast, colon, gastric, lung, and/or ovarian tumor) over the course of treatment) and testing the cancer cells for the level of metabolic and signaling markers to monitor the status of the cancer cells to verify at the molecular level the regression of the cancer cells to a less malignant phenotype. For example, tumor regression induced by employing the methods of this invention would be indicated by finding a decrease in any of the pro-angiogenic markers discussed above, an increase in anti-angiogenic markers described herein, the normalization (i.e., alteration toward a state found in normal individuals not suffering from such cancer) of metabolic pathways, intercellular signaling pathways, or intracellular signaling pathways that exhibit abnormal activity in individuals diagnosed with cancer (e.g., with breast, colon, gastric, lung, and/or ovarian cancer). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

Treat: As used herein, the term “treat,” “treatment,” or “treating” refers to partial or complete alleviation, amelioration, delay of onset of, inhibition, prevention, relief, and/or reduction in incidence and/or severity of one or more symptoms or features of a disease, disorder, and/or condition. In some embodiments, treatment may be administered to a subject who does not exhibit signs or features of a disease, disorder, and/or condition (e.g., may be prophylactic). In some embodiments, treatment may be administered to a subject who exhibits only early or mild signs or features of the disease, disorder, and/or condition, for example for the purpose of decreasing the risk of developing pathology associated with the disease, disorder, and/or condition. In some embodiments, treatment may be administered to a subject who exhibits established, severe, and/or late-stage signs of the disease, disorder, or condition.

Treatment: As used herein, the term “treatment” (also “treat” or “treating”) refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Tumor: As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments, a tumor is associated with, or is a manifestation of, a cancer. In some embodiments, a tumor may be a disperse tumor or a liquid tumor. In some embodiments, a tumor may be a solid tumor.

Variant: As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a small molecule may have a characteristic core structural element (e.g., a macrocycle core) and/or one or more characteristic pendent moieties so that a variant of the small molecule is one that shares the core structural element and the characteristic pendent moieties but differs in other pendent moieties and/or in types of bonds present (single vs double, E vs Z, etc) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. For example, a variant polypeptide may differ from a reference polypeptide as a result of one or more differences in amino acid sequence and/or one or more differences in chemical moieties (e.g., carbohydrates, lipids, etc) covalently attached to the polypeptide backbone. In some embodiments, a variant polypeptide shows an overall sequence identity with a reference polypeptide that is at least 85%, 86%, 87%, 88%, 89%, 90%, 91%, 92%, 93%, 94%, 95%, 96%, 97%, or 99%. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide. In many embodiments, a polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2% of the residues in the variant are substituted as compared with the parent. In some embodiments, a variant has 10, 9, 8, 7, 6, 5, 4, 3, 2, or 1 substituted residue as compared with a parent. Often, a variant has a very small number (e.g., fewer than 5, 4, 3, 2, or 1) number of substituted functional residues (i.e., residues that participate in a particular biological activity). Furthermore, a variant typically has not more than 5, 4, 3, 2, or 1 additions or deletions, and often has no additions or deletions, as compared with the parent. Moreover, any additions or deletions are typically fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, the parent or reference polypeptide is one found in nature. As will be understood by those of ordinary skill in the art, a plurality of variants of a particular polypeptide of interest may commonly be found in nature, particularly when the polypeptide of interest is an infectious agent polypeptide.

### DETAILED DESCRIPTION OF CERTAIN EMBODIMENTS

The present invention provides methods and compositions for the treatment of cancer by targeting CCR8, and/or for identifying and/or characterizing useful cancer therapeutic and/or diagnostic agents that target CCR8.

### Tregs and Immune Evasion

The solid tumor microenvironment contains a variety of immune cells. Extensive human and mouse experimental studies suggest that the types and properties of the immune cells residing within a tumor influence clinical response. In particular, regulatory T (Treg) cell presence is associated with poor clinical outcome in melanoma, breast, gastric, ovarian, pancreatic and other cancer types, while a high CD8+ tumor infiltrating lymphocyte (TIL) density correlates with improved survival in several cancer types. In large cohorts of human colorectal tumors, immunological parameters (type, density, location of immune cells within a tumor) were a better predictor of survival than the current histopathological methods used for staging.

Regulatory T cells (Treg) are a subset of CD4 T cells that are required for control of autoimmunity, dampening excessive inflammation caused by the immune response to pathogens, and maintaining maternal-fetal tolerance. Regulatory T cells (Treg) are important in maintaining homeostasis, controlling the magnitude and duration of the inflammatory response, and in preventing autoimmune and allergic responses. There are two major classifications of Treg: natural Treg and peripheral Treg. Natural Treg, (nTreg) are a class of thymically generated T-cells while peripheral Treg (pTreg) develop in the periphery from naïve T cells in response to signals such as low doses of antigen, presence of certain microbes, lymphopenia or, in some cases, through activation by immature dendritic cells. In some cases, pTreg are thought to be generated in response to inflammatory conditions, particularly those which may be due at least in part to the absence of nTreg cells.

The Forkhead box P3 transcription factor (Foxp3) has been shown to be a key regulator in the differentiation and activity of Treg. In fact, loss-of-function mutations in the Foxp3 gene have been shown to lead to the lethal IPEX syndrome (immune dysregulation, polyendocrinopathy, enteropathy, X-linked). Patients with IPEX suffer from severe autoimmune responses, persistent eczema, and colitis.

In general Tregs are thought to be mainly involved in suppressing immune responses, functioning in part as a “self-check” for the immune system to prevent excessive reactions. In particular, Tregs are involved in maintaining tolerance to self-antigens, harmless agents such as pollen or food, and abrogating autoimmune disease.

Tregs are found throughout the body including, without limitation, the gut, skin, lung, and liver. Additionally, Treg cells may also be found in certain compartments of the body that are not directly exposed to the external environment such as the spleen, lymph nodes, and even adipose tissue. Each of these Treg cell populations is known or suspected to have one or more unique features and additional information may be found in Lehtimaki and Lahesmaa (Regulatory T cells control immune responses through their non-redundant tissue specific features, FRONTIERS IN IMMUNOL., 4 (294): 1-10, 2013), the disclosure of which is hereby incorporated in its entirety.

Typically, regulatory T cells are known to require TGF-β and IL-2 for proper activation and development. Blockade of TGF-β signaling has been shown to result in systemic inflammatory disease as a result of a deficiency of Treg and IL-2 knockout mice have been shown to fail to develop Treg. TGF-β may be particularly important, as it is known to stimulate Foxp3, the transcription factor that drives differentiation of T cells toward the Treg lineage.

Tregs are known to produce both IL-10 and TGF-β, both potent immune suppressive cytokines. Additionally, Tregs are known to inhibit the ability of antigen presenting cells (APCs) to stimulate T cells. One proposed mechanism for APC inhibition is via CTLA-4, which is expressed by Foxp3+ Treg. It is thought that CTLA-4 may bind to B7 molecules on APCs and either block these molecules or remove them by causing internalization resulting in reduced availability of B7 and an inability to provide adequate co-stimulation for immune responses. Additional discussion regarding the origin, differentiation and function of Treg may be found in Dhamne et al., Peripheral and thymic Foxp3+ regulatory T cells in search of origin, distinction, and function, 2013, Frontiers in Immunol., 4 (253): 1-11, the disclosure of which is hereby incorporated in its entirety.

While Tregs are critical for maintaining peripheral tolerance, their potent immunoregulatory properties can promote the development of numerous types of malignancies by inhibiting effector responses. For certain cancers (e.g., breast, colon, gastric, lung, and/or ovarian cancers) the presence of large numbers of Treg cells correlates with poor outcome. Furthermore, clinical evaluation of human breast cancers reveals that the prevalence of Treg among tumor infiltrating lymphocytes increases with disease stage. A decrease in the number of breast tumor infiltrating Tregs is positively associated with a pathological response to neoadjuvant chemotherapy. Previous data reveal that the specific ablation of Treg in advanced murine breast tumors leads to a significant delay in tumor growth and a dramatic reduction in metastatic burden. See Bos P D, et al., J Exp Med 2013; 210(11):2435-66. As described previously, CCR8 is a useful target specific to tumor-infiltrating Treg cells. See Immunity. 2016 Nov. 15; 45(5):1122-1134 and U.S. Pat. No. 10,087,259. In some embodiments, CCR8 may be upregulated in tumor-infiltrating Treg cells relative, for example, to its expression in Tregs resident in otherwise comparable non-tumor tissue(s) or cell(s).

### CCR8

CCR8 is a member of the β-chemokine receptor family, which is predicted to be a seven transmembrane protein similar to G protein-coupled receptors. Chemokines and their receptors are known to be important for the migration of various cell types into the inflammatory sites.

CCR8 has been reported to play a role in regulation of monocyte chemotaxis and thymic cell apoptosis. More specifically, it has been suggested that CCR8 may contribute to proper positioning of activated T cells within antigenic challenge sites and specialized areas of lymphoid tissues.

In humans, the gene encoding CCR8 is located in the chemokine receptor gene cluster region 3p22.

Identified ligands of CCR8 include its natural cognate ligand, CCL1 (aka I-309), thymus activation-regulated cytokine (TARC) and macrophage inflammatory protein-1 beta (MIP-1 beta).

CCR8 is preferentially expressed in the thymus, and recent reports have indicated that its expression is elevated in human cancer tissues, primarily limited to tumor-associated macrophages (see Eruslanov et al, Clin Cancer Res. 19:1670, Epub 2013 Jan. 30). The present disclosure recognizes that, in fact, CCR8 is specifically expressed in Treg cells, and more particularly in tumor-infiltrating Tregs. The present disclosure specifically teaches that CCR8 is specifically expressed in tumor-infiltrating Tregs as compared with other tumor-infiltrating T cell subsets (i.e., tumor infiltrating CD4 and CD8 T cells), and demonstrates that CCR8 can serve as an effective target to mediate depletion of such tumor-infiltrating Treg cells.

### Anti-CCR8 Agents

In light of the teaching that CCR8 can effectively be targeted to achieve specific depletion of Treg cells, and particularly of tumor-infiltrating Treg cells, those skilled in the art will appreciate that any of a variety of appropriate agents may be used to target CCR8 and achieve such depletion.

In some embodiments, an anti-CCR8 agent for use in accordance with the present invention is or comprises an antibody agent. In some embodiments, an antibody agent is or comprises a CCR8-specific antibody or antigen-binding fragment. In some embodiments, an antibody agent is or comprises an antibody or antigen-binding fragment thereof that binds to a CCR8 polypeptide found on the surface of a cell. In some embodiments, an antibody agent is or comprises an antibody or antigen-binding fragment thereof that binds to a CCR8 polypeptide found on the surface of Treg cells. In some embodiments, an antibody agent is or comprises an antibody or antigen-binding fragment thereof that binds to a CCR8 polypeptide found on the surface of Treg cells that have infiltrated a tumor. In some embodiments, an antibody agent is or comprises an antibody or antigen-binding fragment thereof that binds to a human CCR8 polypeptide. In some embodiments, an antibody agent is or comprises an antibody or antigen-binding fragment thereof that binds to a cynomolgus monkey CCR8 polypeptide.

In some embodiments, an anti-CCR8 agent is or comprises an antibody or antigen-binding fragment thereof that competes for binding of CCR8 with any other agent, antibody or ligand. In some embodiments, an anti-CCR8 agent is or comprises an antibody or antigen-binding fragment thereof that competes for binding of CCR8 with CCL1. In some embodiments, an anti-CCR8 agent is or comprises an antibody or antigen-binding fragment thereof that competes for binding of CCR8 with virokine MC148R. In some embodiments, an anti-CCR8 agent is or comprises an antibody or antigen-binding fragment thereof that competes for binding of CCR8 with other known anti-CCR8 binding antibodies or fragments thereof. In some embodiments, an anti-CCR8 agent is or comprises an antibody or antigen-binding fragment thereof that competes for binding of CCR8 with anti-human CCR8 antibody clone L263G8.

In some embodiments, an anti-CCR8 agent is or comprises an antibody or antigen-binding fragment thereof that activates ADCC.

In some embodiments, an anti-CCR8 agent is or comprises an antibody or antigen-binding fragment which comprises a heavy chain comprising one or more of SEQ ID Nos. 1-14. In some embodiments, an anti-CCR8 agent is or comprises an antibody or antigen-binding fragment which comprises a light chain comprising one or more of SEQ ID Nos. 15-25.

In some embodiments, an anti-CCR8 agent is or comprises an antibody or antigen-binding fragment which comprises a variable heavy chain 80%, 85%, 90%, 95%, or 99% sequence identity to SEQ ID NO. 38. In some embodiments, an anti-CCR8 agent is or comprises an antibody or antigen-binding fragment which comprises a variable heavy chain 80%, 85%, 90%, 95%, or 99% sequence identity to SEQ ID NO. 39. In some embodiments, an anti-CCR8 agent is or comprises an antibody or antigen-binding fragment which comprises a variable heavy chain 80%, 85%, 90%, 95%, or 99% sequence identity to SEQ ID NO. 40. In some embodiments, an anti-CCR8 agent is or comprises an antibody or antigen-binding fragment which comprises a variable heavy chain 80%, 85%, 90%, 95%, or 99% sequence identity to SEQ ID NO. 41. In some embodiments, an anti-CCR8 agent is or comprises an antibody or antigen-binding fragment which comprises a variable heavy chain 80%, 85%, 90%, 95%, or 99% sequence identity to SEQ ID NO. 42. In some embodiments, an anti-CCR8 agent is or comprises an antibody or antigen-binding fragment which comprises a variable heavy chain 80%, 85%, 90%, 95%, or 99% sequence identity to SEQ ID NO. 43.

In some embodiments, an anti-CCR8 agent is or comprises an antibody or antigen-binding fragment which comprises a variable light chain 80%, 85%, 90%, 95%, or 99% sequence identity to SEQ ID NO. 44. In some embodiments, an anti-CCR8 agent is or comprises an antibody or antigen-binding fragment which comprises a variable light chain 80%, 85%, 90%, 95%, or 99% sequence identity to SEQ ID NO. 45. In some embodiments, an anti-CCR8 agent is or comprises an antibody or antigen-binding fragment which comprises a variable light chain 80%, 85%, 90%, 95%, or 99% sequence identity to SEQ ID NO. 46. In some embodiments, an anti-CCR8 agent is or comprises an antibody or antigen-binding fragment which comprises a variable light chain 80%, 85%, 90%, 95%, or 99% sequence identity to SEQ ID NO. 47. In some embodiments, an anti-CCR8 agent is or comprises an antibody or antigen-binding fragment which comprises a variable light chain 80%, 85%, 90%, 95%, or 99% sequence identity to SEQ ID NO. 48. In some embodiments, an anti-CCR8 agent is or comprises an antibody or antigen-binding fragment which comprises a variable light chain 80%, 85%, 90%, 95%, or 99% sequence identity to SEQ ID NO. 49.

In some embodiments, an anti-CCR8 agent is or comprises an antibody or antigen-binding fragment which comprises a heavy chain variable domain comprising an amino acid sequence including each of a sequence selected from SEQ ID Nos. 6-9; a sequence selected from SEQ ID Nos. 10-14; a sequence selected from SEQ ID Nos. 1-5; and wherein the light chain variable domain has an amino acid sequence including each of: a sequence selected from SEQ ID Nos. 15-18; a sequence selected from SEQ ID Nos. 19-21; and a sequence selected from SEQ ID Nos. 22-25.

In some embodiments, an anti-CCR8 agent is considered to target CCR8 if it is demonstrated to bind CCR8. In some embodiments, an anti-CCR8 agent is considered to target CCR8 if it competes with a reference anti-CCR8 antibody (e.g., anti-CCR8 clone L263G8) for binding.

In some embodiments, an anti-CCR8 agent is or comprises an antibody or antigen-binding fragment which comprises a sequence or sequences having 80%, 85%, 90%, 95%, or 99% sequence identity to those sequences identified in the Examples included herein.

### Formats of Antibody Agents

A wide variety of formats has been developed for antibody agents, several of which have already progressed into clinical trials (reviewed, for example, in Scott A M et al., 2012). In some embodiments, an antibody or fragment thereof utilized in accordance with the present invention is in a format selected from, but not limited to, intact IgG, IgE and IgM, bi- or multi-specific antibodies (e.g., Zybodies®, etc), single chain Fvs, polypeptide-Fc fusions, Fabs, cameloid antibodies, masked antibodies (e.g., Probodies®), Small Modular ImmunoPharmaceuticals (“SMIPs™”), single chain or Tandem diabodies (TandAb®), VHHs, Anticalins®, Nanobodies®, minibodies, BiTE® s, ankyrin repeat proteins or DARPINs®, Avimers®, a DART, a TCR-like antibody, Adnectins®, Affilins®, Trans-bodies®, Affibodies®, a TrimerX®, MicroProteins, Fynomers®), Centyrins®, CoVX bodies, BiCyclic peptides, or Kunitz domain derived antibody constructs.

In some embodiments, an antibody agent of the present disclosure is a masked antibody (e.g., Probody®). In some embodiments, use of such a format ensures that CCR8 targeting occurs substantially or only in the tumor milieu, and not elsewhere in the body. In some embodiments, use of such a format specifically ensures targeting of CCR8 on Tregs that have infiltrated the tumor.

### Tumors

Technologies provided herein are useful in the treatment of any tumor.

In some embodiments, a tumor is a solid tumor, including but not limited to breast carcinoma, a squamous cell carcinoma, a colon cancer, a head and neck cancer, a lung cancer, a genitourinary cancer, a rectal cancer, a gastric cancer, or an esophageal cancer. In some particular embodiments, a tumor is selected from a lymphoma, a breast tumor, a colon tumor, and a lung tumor.

### Application and Uses

In general, provided anti-CCR8 agents are useful to bind to, and/or to compete with another potential binding partner for binding to, CCR8 (e.g., in or on cells, such as in particular tumor-infiltrating Treg cells). In some embodiments, provided anti-CCR8 agents are useful in the treatment of cancer (e.g., breast, colon, gastric, lung, and/or ovarian cancer). In some embodiments, provided anti-CCR8 agents are useful in the treatment of cancer (e.g., breast, colon, gastric, lung, and/or ovarian cancer) by initiating, causing, or playing a role in depletion of tumor-infiltrating Treg cells.

Alternatively or additionally, in some embodiments, provided anti-CCR8 agents are useful in the development and/or characterization of other CCR8-binding agents.

Technologies provided herein, including provided anti-CCR8 agents and/or components thereof, find application in various contexts, including therapy (e.g., cancer therapy such as therapy for breast, colon, gastric, lung, and/or ovarian malignancy, etc), detection, and drug development.

### Therapeutics

### Pharmaceutical Composition

In some embodiments, the present disclosure provides a pharmaceutical composition that comprises and/or delivers anti-CCR8 agents. In some embodiments a pharmaceutical composition comprises an anti-CCR8 agent that is or comprises an antibody or fragment thereof. In some embodiments a pharmaceutical composition delivers anti-CCR8 agents. In some embodiments, delivery of anti-CCR8 agents comprises administration of a nucleic acid encoding an anti-CCR8 agent. In some embodiments, delivery of anti-CCR8 agents comprises administration of a vector encoding an anti-CCR8 agent. In some embodiments, delivery of anti-CCR8 agents comprises administration of cells expressing an anti-CCR8 agent.

### Administration

In some embodiments, the present disclosure provides for administration of anti-CCR8 agents. In some embodiments, anti-CCR8 agents of the present disclosure are administered in a pharmaceutical composition. In some embodiments, anti-CCR8 agents are administered according to a regimen sufficient to permit binding, eg., to CCR8, in or on cell(s) e.g., tumor infiltrating Tregs. In some embodiments, anti-CCR8 agents are administered according to a regimen sufficient to permit depletion of tumor infiltrating Tregs. In some embodiments, anti-CCR8 agents are administered according to a regimen sufficient to permit or achieve detection of CCR8 in a sample.

### Combination

Those of ordinary skill in the art, reading the present disclosure, will readily appreciate that anti-CCR8 agents, as described herein, may in certain embodiments be combined with other anti-cancer therapies, including for example administration of chemotherapeutic agents, other immunomodulatory agents, radiation therapy, high-frequency ultrasound therapy, surgery, etc.

Thus, in some embodiments, an anti-CCR8 agent, as described herein, is utilized in combination with one or more other therapeutic agents or modalities. In some embodiments, the one or more other therapeutic agents or modalities is also an anti-cancer agent or modality; in some embodiments the combination shows a synergistic effect in treating cancer. For example, as described herein, in some embodiments, therapy with an anti-CCR8 agent is combined with anti-tumor antibody therapy.

Known compounds or treatments that show therapeutic efficacy in treating cancer may include, for example, one or more alkylating agents, anti-metabolites, anti-microtubule agents, topoisomerase inhibitors, cytotoxic antibiotics, angiogenesis inhibitors, immunomodulators, vaccines, cell-based therapies (e.g. allogeneic or autologous stem cell transplantation), organ transplantation, radiation therapy, surgery, etc.

### Formulation and Administration

Pharmaceutical compositions (e.g., comprising an anti-CCR8 agent, an anti-tumor antibody and/or any other therapeutically active agent) for use in accordance with the present invention may be prepared for storage and/or delivery using any of a variety of techniques and/or technologies known and/or available to those skilled in the art.

In some embodiments, utilized agents (e.g., an anti-CCR8 agent, anti-tumor antibody, and/or any other therapeutically active agent utilized in accordance with the present invention) is administered according to a dosing regimen approved by a regulatory authority such as the United States Food and Drug Administration (FDA) and/or the European Medicines Agency (EMEA), e.g., for the relevant indication. Those skilled in the art will be aware, or will readily be able to determine, approved dosing regimens for a variety of agents, including for example, a variety of anti-tumor antigen antibodies.

Those skilled in the art, reading the present disclosure will appreciate various modifications of dosing regimens that are within the scope of the present invention. For example, just to name a few, in some embodiments, an anti-CCR8 agent is utilized as monotherapy. In some such embodiments, addition of one or more anti-cancer therapy (ies) may be particularly useful.

In some embodiments, dosing and administration according to the present invention utilizes active agents having a desired degree of purity combined with one or more physiologically acceptable carriers, excipients or stabilizers in any or variety of forms. These include, for example, liquid, semi-solid and solid dosage forms, such as liquid solutions (e.g., injectable and infusible solutions), dispersions or suspensions, tablets, pills, powders, liposomes and suppositories. In some embodiments, a preferred form may depend on the intended mode of administration and/or therapeutic application. Typical preferred compositions are in the form of injectable or infusible solutions, such as compositions similar to those used for passive immunization of humans with other antibodies.

In some embodiments, ingredient(s) can be prepared with carriers that protect the agent(s) against rapid release and/or degradation, such as a controlled release formulation, including implants, transdermal patches, and microencapsulated delivery systems. Biodegradable, biocompatible polymers can be used, such as polyanhydrides, polyglycolic acid, polyorthoesters, and polylactic acid.

In general, each active agent is formulated, dosed, and administered in therapeutically effective amount using pharmaceutical compositions and dosing regimens that are consistently with good medical practice and appropriate for the relevant agent(s) (e.g., for agents such as antibodies). Pharmaceutical compositions containing active agents can be administered by any appropriate method known in the art, including, without limitation, oral, mucosal, by-inhalation, topical, buccal, nasal, rectal, or parenteral (e.g. intravenous, infusion, intratumoral, intranodal, subcutaneous, intraperitoneal, intramuscular, intradermal, transdermal, or other kinds of administration involving physical breaching of a tissue of a subject and administration of the pharmaceutical composition through the breach in the tissue).

In some embodiments, a dosing regimen for a particular active agent may involve intermittent or continuous (e.g., by perfusion or other slow release system) administration, for example to achieve a particular desired pharmacokinetic profile or other pattern of exposure in one or more tissues or fluids of interest in the subject receiving therapy.

In some embodiments, different agents administered in combination may be administered via different routes of delivery and/or according to different schedules. Alternatively or additionally, in some embodiments, one or more doses of a first active agent is administered substantially simultaneously with, and in some embodiments via a common route and/or as part of a single composition with, one or more other active agents.

Factors to be considered when optimizing routes and/or dosing schedule for a given therapeutic regimen may include, for example, the particular cancer being treated (e.g., type, stage, location, etc), the clinical condition of a subject (e.g., age, overall health, etc.), the site of delivery of the agent, the nature of the agent (e.g. an antibody or other protein-based compound), the mode and/or route of administration of the agent, the presence or absence of combination therapy, and other factors known to medical practitioners.

Those skilled in the art will appreciate, for example, that route of delivery (e.g., oral vs intravenous vs subcutaneous vs intratumoral, etc) may impact dose amount and/or required dose amount may impact route of delivery. For example, where particularly high concentrations of an agent within a particular site or location (e.g., within a tumor) are of interest, focused delivery (e.g., in this example, intratumoral delivery) may be desired and/or useful.

Those skilled in the art will further appreciate that some embodiments of combination therapies provided in accordance with the present invention achieve synergistic effects: in some such embodiments, dose of one or more agents utilized in the combination may be materially different (e.g., lower) and/or may be delivered by an alternative route, than is standard, preferred, or necessary when that agent is utilized in a different therapeutic regimen (e.g., as monotherapy and/or as part of a different combination therapy).

In some embodiments, one or more features of a particular pharmaceutical composition and/or of a utilized dosing regimen may be modified over time (e.g., increasing or decreasing amount of active in any individual dose, increasing or decreasing time intervals between doses, etc.), for example in order to optimize a desired therapeutic effect or response (e.g., an ADCC response or other biological response that is related to the relevant disease (e.g., cancer such as breast, colon, gastric, lung, and/or ovarian malignancy, etc)-specific immune response).

In general, type, amount, and frequency of dosing of active agents in accordance with the present invention in governed by safety and efficacy requirements that apply when relevant agent(s) is/are administered to a mammal, preferably a human. In general, such features of dosing are selected to provide a particular, and typically detectable, therapeutic response as compared with what is observed absent therapy. In context of the present invention, an exemplary desirable therapeutic response may involve, but is not limited to, inhibition of and/or decreased tumor growth, tumor size, metastasis, one or more of the symptoms and side effects that are associated with the tumor, as well as increased apoptosis of cancer cells. Such criteria can be readily assessed by any of a variety of immunological, cytological, and other methods that are disclosed in the literature. In particular, the therapeutically effective amount of an anti-CCR8 agent, alone or in combination with a further agent, can be determined as being sufficient to enhance killing of cancer cells (e.g., breast, colon, gastric, lung, and/or ovarian malignancy, etc.).

A therapeutically effective amount of an active agent or composition comprising it can be readily determined using techniques available in the art including, for example, considering one or more factors such as the disease or condition being treated, the stage of the disease, the age and health and physical condition of the mammal being treated, the severity of the disease, the particular compound being administered, and the like.

In some embodiments, therapeutically effective amount is an effective dose (and/or a unit dose) of an active agent that may be at least about 0.01 Mg/kg body weight, at least about 0.05 Mg/kg body weight; at least about 0.1 Mg/kg body weight, at least about 1 Mg/kg body weight, at least about 2.5 Mg/kg body weight, at least about 5 Mg/kg body weight, and not more than about 100 Mg/kg body weight. It will be understood by one of skill in the art that in some embodiments such guidelines may be adjusted for the molecular weight of the active agent. The dosage may also be varied for route of administration, the cycle of treatment, or consequently to dose escalation protocol that can be used to determine the maximum tolerated dose and dose limiting toxicity (if any) in connection to the administration of the first agent, second agent, and/or the third agent at increasing doses. Consequently, the relative amounts of each agent within a pharmaceutical composition may also vary, for example, each composition may comprise between 0.001% and 100% (w/w) of the corresponding agent.

Therapeutic compositions typically should be sterile and stable under the conditions of manufacture and storage. The composition can be formulated as a solution, microemulsion, dispersion, liposome, or other ordered structure suitable to high drug concentration. Sterile injectable solutions can be prepared by incorporating the antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze drying that yields a powder of the active ingredient plus any additional desired ingredient from a previously sterile filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The formulation of each agent should desirably be sterile, as can be accomplished by filtration through sterile filtration membranes, and then packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations as discussed herein. Sterile injectable formulations may be prepared using a non-toxic parenterally acceptable diluent or solvent, such as water or 1,3 butanediol, for example. Other parentally-administrable formulations which are useful include those which comprise the active ingredient in microcrystalline form, in a liposomal preparation, or as a component of biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Each pharmaceutical composition for use in accordance with the present invention may include pharmaceutically acceptable dispersing agents, wetting agents, suspending agents, isotonic agents, coatings, antibacterial and antifungal agents, carriers, excipients, salts, or stabilizers are non-toxic to the subjects at the dosages and concentrations employed. A non-exhaustive list of such additional pharmaceutically acceptable compounds includes buffers such as phosphate, citrate, and other organic acids; antioxidants including ascorbic acid and methionine; salts containing pharmacologically acceptable anions (such as acetate, benzoate, bicarbonate, bisulfate, isothionate, lactate, lactobionate, laurate, malate, maleate, salicylate, stearate, subacetate, succinate, tannate, tartrate, teoclate, tosylate, thiethiodode, and valerate salts); preservatives (such as octadecyidimethylbenzyl ammonium chloride; hexamethonium chloride; benzalkonium chloride, benzethonium chloride; sodium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or antibodies; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG). [134]. In some embodiments, where two or more active agents are utilized in accordance with the present invention, such agents can be administered simultaneously or sequentially. In some embodiments, administration of one agent is specifically timed relative to administration of another agent. For example, in some embodiments, a first agent is administered so that a particular effect is observed (or expected to be observed, for example based on population studies showing a correlation between a given dosing regimen and the particular effect of interest).

In some embodiments, desired relative dosing regimens for agents administered in combination may be assessed or determined empirically, for example using ex vivo, in vivo and/or in vitro models: in some embodiments, such assessment or empirical determination is made in vivo, in a patient population (e.g., so that a correlation is established), or alternatively in a particular patient of interest.

In some embodiments, one or more active agents utilized in practice of the present invention is administered according to an intermittent dosing regimen comprising at least two cycles. Where two or more agents are administered in combination, and each by such an intermittent, cycling, regimen, individual doses of different agents may be interdigitated with one another. In some embodiments, one or more doses of the second agent is administered a period of time after a dose of the first agent. In some embodiments, each dose of the second agent is administered a period of time after a dose of the first agent. In some embodiments, each dose of the first agent is followed after a period of time by a dose of the second agent. In some embodiments, two or more doses of the first agent are administered between at least one pair of doses of the second agent: in some embodiments, two or more doses of the second agent are administered between al least one pair of doses of the first agent. In some embodiments, different doses of the same agent are separated by a common interval of time: in some embodiments, the interval of time between different doses of the same agent varies. In some embodiments, different doses of the different agents are separated from one another by a common interval of time; in some embodiments, different doses of the different agents are separated from one another by different intervals of time.

### Detection

In some embodiments, the present disclosure provides anti-CCR8 agents useful for the detection of CCR8. In some embodiments, the present disclosure provides anti-CCR8 agents useful for the detection of CCR8 in a sample. In some embodiments, a sample can be a biological sample. In some embodiments, a biological sample can be a sample for a subject. In some embodiments a biological sample can be a sample from a tumor in a subject (e.g., a biopsy).

In some embodiments anti-CCR8 agents of the present disclosure further comprises a detectable moiety (e.g., a detectable entity). In some embodiments, a detectable moiety is covalently attached to an anti-CCR8 agent (e.g., via linker, which may in some embodiments be a bond). In some embodiments a detectable moiety is a fluorescent moiety, a radioactive moiety, or an enzymatic moiety. One of skill in the art will recognize many detectable moieties available in the art.

### EXEMPLIFICATION

### Example 1: Materials and Methods for Identification and/or Characterization of Certain Anti-CCR8 Agents as Described Herein

The present Example demonstrates methods for identifying and/or characterizing certain anti-CCR8 agents-specifically particular antibodies and/or antigen binding elements thereof that bind CCR8 as described herein. The present Example further provides various methods for determining and/or characterizing relevant functional activity of antibody agents described herein.

### Antibody Discovery

Antibodies and/or antigen binding elements as described herein were identified from screens of yeast antibody expression libraries and lymphocytes generated by mouse immunizations.

### Mouse Immunization

Humanized anti-CCR8 antibodies were generated by immunizing mice with a plasmid expressing human CCR8 and a cell line expressing human CCR8, and then isolating lymphocytes expressing CCR8-specific antibodies from such immunized mice.

### Antigen and Immunization

Human CCR8antigen, prepared using standard recombinant technology techniques using GenBank sequence as reference (NM_005201.4), was used for immunization.

CCR8-expressing DNA plasmid was delivered into the hind leg of anesthetized 6-8 week old BALB/c mice by injection and electroporation. After this DNA immunization, immune responses were boosted by intraperitonial (IP) administration of ˜10⁶ cells expressing CCR8. At various time points, mice were bled for plasma to assay the immune response that had developed to the antigen.

### Recovery of Lymphocytes and FACSs Sorting

Immunized mice were individually euthanized using CO₂ asphyxiation and cervical dislocation. Spleen and accessible lymph nodes were collected and B cells were mechanically dissociated from lymphoid tissue by grinding in PBS to release the cells from tissues. Cells were suspended in PBS prior to further separation using centrifugation.

Cells were then stained for 30 min with antibody cocktail on ice. Cells were spun down at 300 g for 10 min and were resuspended with FACS buffer. Cells were washed once more and then B cells were single-cell sorted using a BD FACS Fusion (BD Biosciences). Cells were sorted into 96-well PCR plates (BioRAD) containing 20 μL/well of lysis buffer [5 μL of 5× first strand cDNA buffer (Invitrogen), 0.625 μL of NP-40 (New England Biolabs), 0.25 μL RNaseOUT (Invitrogen), 1.25 L dithiothreitol (Invitrogen), and 12.6 μL dH2O]. Plates were immediately stored at −80° C.

### Amplification and Cloning of Antibody Variable Genes

Antibody variable genes (IgH and IgK) were amplified by reverse transcription PCR and nested PCRs using cocktails of IgG- and IgM-specific primers, as described previously (Tiller et al. 2009 J Immunol Methods 350:183-93). The primers used in the second round of PCR contained 40 base pairs of 5′ and 3′ homology to the digested expression vectors, which allowed for cloning by homologous recombination into S. cerevisiae. The lithium acetate method for chemical transformation was used to clone the PCR products into S. cerevisiae (Gietz and Schiestl, Nat Protoc 2007). 10 μL of unpurified heavy chain and light chain PCR product and 200 ng of the digested expression vectors were used per transformation reaction. Following transformation, individual yeast clones were sorted for expression and then colonies picked for sequencing and characterization.

### Expression and Purification of IgGs

IgGs were expressed in S. cerevisiae cultures grown in 24-well plates, as described by Bornholdt et al. 2016 Science 351:1078-83. After 6 days, the cultures were harvested by centrifugation and IgGs were purified by protein A-affinity chromatography. The bound antibodies were eluted with 200 mM acetic acid/50 mM NaCl (pH 3.5) into ⅛th volume 2 M Hepes (pH 8.0), and buffer-exchanged into PBS (pH 7.0).

### Yeast Antibody Library Screens

### Antigen Preparation

CHO cells expressing human CCR8 (CHO-CCR8) or CHO cells stably transfected with an empty vector (CHO-EV) were biotinylated using the EZ-Link Sulfo-NHS-Biotinylation Kit (Thermo Scientific, Cat #21425). In brief, cells were resuspended into PBS to a density of 2.5×10⁷ cells/ml before addition of a 10 mg/mL stock solution of biotinylation reagent to a final dilution of 0.1 mg/mL. The cell-biotin suspension was held at 4° C. for 15 minutes with rotation. Following incubation, cells were washed three times at 500×g for 5 minutes in PBS containing 0.1% bovine serum albumin (BSA), otherwise known as PBSF, to remove free biotin in the solution. Cells were reconstituted in selection buffer (PBS supplemented with 2% BSA and 2 mM EDTA) to a final concentration of 2×10⁷ cells/ml. Biotinylation was confirmed by flow cytometry utilizing EA-PE as a detection reagent.

### Naïve Library Selections

A single naïve, human synthetic, yeast library of ˜10⁹ diversity was propagated as described in, e.g., Y. Xu et al, PEDS 26(10), 663-70 (2013); WO2009036379; WO2010105256; and WO2012009568.

This library was subjected to four rounds of selection using biotinylated mammalian cell:yeast panning in conjunction with two complementary magnetic bead sorting techniques to enrich for target-specific binders. In round one (R1), library pre-clears were performed by incubating 4×10⁹ cells per library with 8×10⁷ biotinylated CHO-EV cells for 20 minutes at room temperature with rotation in selection buffer. Following incubation, Streptavidin MicroBeads (2 mL) were added to the cell-yeast mixture and further incubated for 15 minutes at 4° C. with rotation. Next, the mixture was loaded onto Miltenyi LS columns inserted into a magnetic QuadroMACS Separator and washed three times with 3 mL selection buffer. The eluate from the columns (CHO cell non-binders; aka pre-cleared library) was collected and incubated with 3×10⁷ biotinylated CHO-CCR8 cells for 20 minutes at room temperature with rotation in selection buffer. Following incubation, Streptavidin MicroBeads (1 mL) were added to the cell-yeast mixture and further incubated for 15 minutes at 4° C. with rotation. Next, the mixture was loaded onto Miltenyi LS columns inserted into a magnetic QuadroMACS Separator and washed three times with 3 ml selection buffer. Columns were subsequently removed from the magnet and captured complexes were eluted into flasks containing 200 mL of yeast growth media for propagation.

Round 2 (R2) was performed identically to R1 with the following changes: 1e9 yeast were pre-cleared with 4e7 biotinylated CHO-EV cells using Miltenyi LS columns and 2.5 mL Streptavidin MicroBeads. Remaining yeast after pre-clear were incubated with 4e7 biotinylated CHO-CCR8 cells, Miltenyi LS columns and 2.0 mL Streptavidin MicroBeads.

Round 3 (R3) preclear was performed identically to R1 with the following changes: 2e9 yeast were pre-cleared with 1e8 biotinylated CHO-EV cells using Miltenyi LS columns and 5.0 mL Streptavidin MicroBeads. After the pre-clear was completed, 1e9 remaining yeast were incubated with 1e8 biotinylated CHO-CCR8 cells for 15 minutes at 4° C. with rotation after which 1 mL of pre-washed M-280 Strepavidin Dynabeads (Cat #60210) were added to the yeast/mammalian cell complexes and incubated for 20 minutes at 4° C. Next, the complexes were separated using a DynaMag-2 magnet and the non-binding supernatants were removed. The bead/cell complexes were washed three times with 1 mb of selection buffer. The captured complexes were then transferred into flasks containing yeast growth media for propagation.

In Round 4 (R4), 1e9 yeast were positively selected on 1e8 biotinylated CHO-EV cells in one instance. In another instance, 1e9 yeast were positively selected on 1e8 biotinylated CHO-CCR8 cells. The CHO-EV/yeast and CHO-CCR8/yeast complexes were allowed to incubate for 15 minutes at 4° C. with rotation after which 1 mL of pre-washed M-280 Strepavidin Dynabeads (Cat #60210) were added to the yeast/mammalian cell complexes and incubated for an additional 20 minutes at 4° C. Next, the complexes were separated using a DynaMag-2 magnet and the non-binding supernatants were removed. The bead/cell complexes were washed three times with 1 mL of selection buffer. The captured complexes were then transferred into flasks containing yeast growth media for propagation. After this final round of selection, yeast were dilution-plated and individual colonies were picked for characterization. Alternatively, selection outputs were subjected to Next Generation Sequencing (NGS) and bioinformatics analyses to identify CCR8-specific sequences as described below.

### Next Generation Sequencing

Antibody heavy chains from R3 and R4 selection outputs were used to prepare barcoded DNA libraries and multiplexed for analysis by Illumina Miseq to query frequency changes predictive of target-specific binding.

Heavy chain plasmids were extracted from the R3 CHO-CCR8 output, R4 CHO-EV output and R4 CHO-CCR8 output. Heavy chain DNA was quantified against a standard curve using SsoFast 2X EvaGreen Supermix (BioRad) and a Bio-Rad CFX96 Real-Time PCR Detection System Thermocycle. Once quantified, NGS amplicon libraries were generated through the addition of Illumina nucleotide barcode sequences by PCR. Following PCR, products were purified via Aline PCRClean DX SPRI beads (Cat #C-1003) and subjected to 6% TBE PAGE for size selection. Full length-VH fragments were excised from the gel and DNA was recovered though incubation in 300 mM sodium acetate pH5.5 and 1 mM EDTA followed by standard ethanol precipitation. Purified heavy chain DNA was quantified using a Kapa Library Quantification kit from Kapa Biosystems (Cat #KK4824). Samples were mixed to generate a multiplexed barcoded pool at greater than 2 nM concentration and submitted for paired end Next Generation Sequencing using the Illumina MiSeq platform and MiSeq Reagent Kit v3 (Cat #MS-102-3003). Raw data from the MiSeq was analyzed via proprietary software developed by Adimab LLC. In brief, paired-end DNA reads were assembled, aggregated and subjected to binomial statistical analysis. Enrichment of unique VH sequences was calculated based upon frequency changes between any given selection output and its corresponding input. VH sequences demonstrating CHO-CCR8 specific enrichment were subsequently ordered as gBlocks (IDT DNA) and transformed into yeast containing the light chain plasmid of the parent.

### Light Chain Diversification

Heavy chains from naïve output were used to prepare light chain diversification libraries used for additional selection rounds.

Heavy chain plasmids were extracted from the yeast, propagated in and subsequently purified from E. coli, and transformed into a light chain library with a diversity of ˜5×10⁶. Selections were performed on these libraries as described for the naïve discovery with slight modifications as described below.

Round 1 (R1): 1e9 yeast were pre-cleared with 4e7 biotinylated CHO-EV cells using Miltenyi LS columns and 500 μL Streptavidin MicroBeads. Post pre-clear, 5e8 remaining yeast were subsequently incubated with 2.5e7 biotinylated CHO-CCR8 cells for 15 minutes at 4° C. with rotation after which 500 μL of pre-washed M-280 Strepavidin Dynabeads (Cat #60210) were added to the yeast/mammalian cell complexes and incubated for 20 minutes at 4° C. Next, the complexes were separated using a DynaMag-2 magnet and the non-binding supernatants were removed. The bead/cell complexes were washed three times with 1 mL of selection buffer. The captured complexes were then transferred into flasks containing yeast growth media for propagation.

Round 2 (R2) was identical to R1 with the following changes: 1e9 yeast were pre-cleared with 5e7 biotinylated CHO-EV cells. Post pre-clear, 5e8 remaining yeast were subsequently incubated with 5e7 biotinylated CHO-CCR8 cells.

Round 3 (R3) was performed by using flow cytometry to counter-select against poly-specificity reagent (PSR) binding as described in Y. Xu et al, PEDS 26.10, 663-70 (2013). Sorting was performed using a FACS ARIA sorter (BD Biosciences) and yeast propagated in growth media for additional selection.

Round 4 (R4) was performed identically to R2. After this final round of selection, yeast were dilution plated and individual colonies were picked for characterization.

### Antibody Optimization

Optimization of antibodies was performed by introducing diversities into the heavy chain variable regions as described below.

CDRH2 selection: CDRH2 diversification was obtained by ordering oligos with variegation in the CDRH2. The variable regions (FR1-FR2, FR3-FR4) from the best IgGs of the light chain diversification cycle were combined with the CDRH3 oligos and transformed into yeast containing the light chain plasmid of the parent.

CDRH3 selection: CDRH3 diversification was obtained by ordering oligos with variegation in the CDRH3. The variable regions (FR1-FR3) from the best IgGs of the light chain diversification cycle were combined with the CDRH3 oligos and transformed into yeast containing the light chain plasmid of the parent.

For all optimization cycles, the libraries were assessed for antibody expression and PSR binding after which yeast were dilution plated and individual colonies were picked for characterization.

### Antibody Production and Purification

Yeast clones were grown to saturation and then induced for 48 h at 30° C. with shaking. After induction, yeast cells were pelleted and the supernatants were harvested for purification. IgGs were purified using a Protein A column and eluted with acetic acid, pH 2.0. Fab fragments were generated by papain digestion and purified over KappaSelect (GE Healthcare LifeSciences).

### Mammalian Expression of Antibodies

Alternatively, mammalian expression of IgG was performed by sub-cloning antibodies into a new expression vector followed by transient transfection and expression in HEK. Briefly, expression vectors containing the VH and VL of the antibody of interest were transfected by complexing with a transfection reagent followed by exposure to HEK cells for one hour followed by dilution of culture media to a final density of 4 million cells per mL. The cells were then cultured for 6 days with fresh media feed every 48 hours. After 6 days, the supernatant was harvested by centrifugation and passed over Protein A agarose (MabSelect SuRe; GE Healthcare Life Sciences). The bound antibodies were then washed with PBS and eluted with buffer (200 mM acetic acid/50 mM NaCl, pH 3.5) into ⅛ volume 2 M Hepes, pH 8.0. The final products were buffer-exchanged into 25 mM HEPES and 150 mM sodium chloride, pH 7.3.

### Polyreactivity Assay

Polyspecificity reagent (PSR) binding was assessed as previously described (Xu et al. 2013 Protein Eng Des 26:663-70). Briefly, soluble membrane protein (SMP) and soluble cytosolic protein (SCP) fractions were prepared from CHO cells and biotinylated with NHS-LC-Biotin reagent (Pierce, ThermoFisher Cat #21336). 2 million IgG-presenting yeast were transferred to a 96-well assay plate, pelleted to remove supernatant, then the pellets were resuspended in 50 μL of 1:10 diluted stock of biotinylated SCPs and SMPs and incubated on ice for 20 min. Cells were washed twice with ice-cold PBSF, and the samples were incubated in 50 μL of secondary labeling mix (Extravadin-R-PE, goat F(ab) 2-anti human kappa-FITC, and propidium iodide) on ice for 20 min. The samples were analyzed for polyspecificity reagent binding using a FACSCanto II (BD Biosciences) with HTS sample injector. Flow cytometry data were analyzed for mean fluorescence intensity (MFI) in the R-PE channel and normalized to three control antibodies exhibiting low, medium, and high MFI values. Thus, the PSR score is determined by normalizing values against a set of control IgGs.

### Hydrophobic Interaction Chromatography (HIC)

The methodology for this assay was described, for example, in Xu Y, et al. (2013) Protein Eng Des Sel 26(10):663-670. In brief, 5 μg IgG samples (1 mg/mL) were spiked in with a mobile phase A solution (1.8 M ammonium sulfate and 0.1 M sodium phosphate at pH 6.5) to achieve a final ammonium sulfate concentration of about 1 M before analysis. A Sepax Proteomix HIC butyl-NP5 column was used with a liner gradient of mobile phase A and mobile phase B solution (0.1 M sodium phosphate, pH 6.5) over 20 min at a flow rate of 1 mL/min with UV absorbance monitoring at 280 nm. A clean to low HIC is considered to be approximately less than 10.5 minutes.

### Affinity-Capture Self-Interaction Nanoparticle Spectroscopy

The methodology for this assay was described, for example, in Liu et al. MAbs. 2014 Mar. 1; 6(2): 483-492. A Δλmax<5.0 nm implies low self-interaction.

### CCR8 Binding

Estimation of apparent antibody affinity was assessed by Fab titration on recombinant human CCR8 expressing CHO cells and determination of EC50 values. In brief, serial dilutions of Fab were prepared from 1000 nM to 1 nM and incubated with 50,000 CHO-CCR8 cells on ice for two hours. After primary Fab incubation, cells were washed followed by addition of anti-F(ab)2 conjugated to AlexaFluor 647 secondary reagent (Jackson ImmunoResearch 109-605-006) for 15 minutes on ice in the dark. Wash steps were repeated, and cells were subjected to flow cytometric analysis (BD FACSCANTO). Median fluorescence data were plotted using GraphPad Prism. EC50 values were determined within Prism using the equation Y=((bmax−b)*(x/(K+x)))+b where Y=observed MFI, bmax=maximal observed MFI, b=background, K=KD as observed on cells and x=Fab concentration.

### CCL1 Antagonism

Activity of the antibodies and controls was tested via the use of two (2) biosensors that were robustly engaged following stimulation with hCCL1 (i.e., Gαz and Gα15/16) using commercially available bioSensAll BRET based G protein activation assay (http://biosensall.com/biosensall/). Briefly, Gα plasma membrane (GAPL) biosensors are used to monitor the activation of heterotrimeric G proteins at the plasma membrane upon receptor stimulation. Specifically, these multimolecular BRET sensors detect the plasma membrane recruitment of proteins that interact with active Gα subunits in a G protein family-selective manner. G protein activation following receptor stimulation generally leads to an increase in the BRET signal.

### ADCC

Ability of antibodies to induce ADCC was tested using commercially available kits. Briefly, test antibodies were bound to target cells expressing CCR8. The target cells were then incubated with ADCC effector cells expressing a high or low affinity Fc gamma receptor and a NFAT response element regulating expression of a luciferase reporter. Thus, activation of NFAT signaling through the Fc gamma receptor produces as detectable signal and is indicative of ADCC activity activation.

### Human T-Reg Binding Assay

Lymphocytes from tumor and normal tissues were isolated by mincing the freshly obtained surgical specimens and subsequent enzymatic digestion using Liberase TL (Sigma) for 20 min at 37 deg C. as described, for example, in Plitas et al, Cell, 2016. After passing through a 100 um filter cells were washed twice with PBS prior to staining. PBMC were first enriched for CD4 T cells through negative selection with RosetteSep antibody cocktails (Stem Cell Technologies). Lymphocytes were stained at 1×106 cells per ml for 20 min. Definitions used for cell sorting were for conventional CD4+ T cells: CD45+CD3+CD4+CD8−CD25−; for Treg cells: CD45+CD3+CD4+CD8−CD127−CD25high. Tissue Treg cells were largely of an activated phenotype and for a fair comparison to their peripheral blood counterparts activated Treg cells were defined as CD45+CD3+CD4+CD8−CD45RO+CD127−CD25high, while resting Treg cells were defined as CD45+CD3+CD4+CD8−CD45RA+CD127−CD25high. Post sort purity was routinely >95% pure for the sorted populations. All antibodies were purchased from eBioscience or BioLegend. Stained cells were either analyzed on a LSRII flow cytometer (BD) or sorted using a FACSAria II (BD). Flow cytometry data were analyzed with FlowJo software (TreeStar).

### Example 2: Anti-CCR8 Agents and Activity

The present Example demonstrates antibody agents and relevant functional activity of antibody agents described herein.

Through the screening methods described above numerous antibodies were discovered. Heavy chain variable domain sequences of exemplary such antibodies are presented below in Table 1.

TABLE 1
Variable Heavy Chain Amino Acid Sequence

| SEQ. | | |
|---|---|---|
| ID. | ADI | |
| NO. | Name | VH Protein Sequence |
| 38 | ADI- | QVQLVESGGGVVQPGRSLRLSCAASGFTFSSYGMHWVRQAPG |
| | 40306 | KGLEWVAVISYDGSNKYYAFSVKGRFTISRDNSKNTLYLQMN |
| | | SLRVEDTAVYYCARVRRIAGRAGYGMDVWGQGTTVTVSS |
| 39 | ADI- | QVQLQESGPGLVKPSETLSLTCAVSGYSISSGYYWGWIRQPPG |
| | 40327 | KGLEWIGSIYHSGNTYYRPSLKSRVTISVDTSKNQFSLKLSSVT |
| | | AADTAVYYCARGKGGSWTAFGPWGQGTLVTVSS |
| 40 | ADI- | QLQLQESGPGLVKPSETLSLTCTVSGGSISSSSYAWGWIRQPPG |
| | 40352 | KRLEWIGSIYYTGSTYYNPSLKSRVTISVDTSKNQFSLKLSSVT |
| | | AADTAVYYCLRGHRRDYIAFDIWGQGTMVTVSS |
| 41 | ADI- | QLQLQESGPGLVKPSETLSLTCTVSGGSISSSSYAWGWIRQPPG |
| | 40360 | KRLEWIGSIYYTGSTYYNPSLKSRVTISVDTSKNQFSLKLSSVT |
| | | AADTAVYYCVRGHRRDYIAFDIWGQGTMVTVSS |
| 42 | ADI- | EVQLVESGGGLVQPGGSLRLSCAASGFTFNAYAMNWVRQAP |
| | 46663 | GKGLEWVARIRSKSNNYATYYADSVKDRFTISRDDSKNSLYL |
| | | QMNSLKTEDTAVYYCVRQSYGNSNYAMDYWGQGTTVTVSS |
| 43 | ADI- | EVKLEQSGGGLVQPKGSLKLSCVASGFTFNAYAMNWVRQAP |
| | 35281 | GQGLEWVARIRSKSNNYATYYAASVKDRFTISRDDSQSILYLQ |
| | | MNNLKTEDTAMYYCVRQSYGNSNYAMDYWGQGTTVTVSS |

As those skilled in the art are aware, more than one strategy for defining “framework region” (“FR”) and/or “complementarity determining region” (“CDR”) sequence elements within an antibody variable domain is available, and precise boundaries of such sequence elements as defined by different approaches may vary somewhat. Using one such available strategy, the present disclosure describes FR and CDR sequences within these exemplified HC variable domains as set forth below in Tables 2-8:

TABLE 2
VH-CDR3:

| SEQ. ID. NO. | ADI Name | VH CDR3 Sequence |
|---|---|---|
| 1 | ADI-40306 | ARVRRIAGRAGYGMDV |
| 2 | ADI-40327 | ARGKGGSWTAFGP |
| 3 | ADI-40352 | LRGHRRDYIAFDI |
| 4 | ADI-40360 | VRGHRRDYIAFDI |
| 5 | ADI-46663 | VRQSYGNSNYAMDY |
| | ADI-35281 | |

TABLE 3
VH-CDR1:

| SEQ. ID. NO. | ADI Name | VH CDR1 Sequence |
|---|---|---|
| 6 | ADI-40306 | FTFSSYGMH |
| 7 | ADI-40327 | YSISSGYYWG |
| 8 | ADI-40352 | GSISSSSYAWG |
| | ADI-40360 | |
| 9 | ADI-46663 | FTFNAYAMN |
| | ADI-35281 | |

TABLE 4
VH-CDR2:

| SEQ. ID. NO. | ADI Name | VH CDR2 Sequence |
|---|---|---|
| 10 | ADI-40306 | VISYDGSNKYYAFSVKG |
| 11 | ADI-40327 | SIYHSGNTYYRPSLKS |
| 12 | ADI-40352 | SIYYTGSTYYNPSLKS |
| | ADI-40360 | |
| 13 | ADI-46663 | RIRSKSNNYATYYADSVKD |
| 14 | ADI-35281 | RIRSKSNNYATYYAASVKD |

TABLE 5
VH-Framework Region 1 (FR1):

| SEQ. | | |
|---|---|---|
| ID. NO. | ADI Name | FR1 Sequence |
| 50 | ADI-40306 | QVQLVESGGGVVQPGRSLRLSCAASG |
| 51 | ADI-40327 | QVQLQESGPGLVKPSETLSLTCAVSG |
| 52 | ADI-40352 | QLQLQESGPGLVKPSETLSLTCTVSG |
| | ADI-40360 | |
| 53 | ADI-46663 | EVQLVESGGGLVQPGGSLRLSCAASG |
| 54 | ADI-35281 | EVKLEQSGGGLVQPKGSLKLSCVASG |

TABLE 6
VH-Framework Region 2 (FR2):

| SEQ. ID. NO. | ADI Name | FR2 Sequence |
|---|---|---|
| 55 | ADI-40306 | WVRQAPGKGLEWVA |
| 56 | ADI-40327 | WIRQPPGKGLEWIG |
| 57 | ADI-40352 | SIYYTGSTYYNPSLKS |
| | ADI-40360 | |
| 58 | ADI-46663 | RIRSKSNNYATYYADSVKD |
| 59 | ADI-35281 | WVRQAPGQGLEWVA |

TABLE 7
VH-Framework Region 3 (FR3):

| SEQ. ID. NO. | ADI Name | FR3 Sequence |
|---|---|---|
| 55 | ADI-40306 | RFTISRDNSKNTLYLQMNSLRVEDTAVYYC |
| 56 | ADI-40327 | RVTISVDTSKNQFSLKLSSVTAADTAVYYC |
| | ADI-40352 | |
| | ADI-40360 | |
| 57 | ADI-46663 | RFTISRDDSKNSLYLQMNSLKTEDTAVYYC |
| 58 | ADI-35281 | RFTISRDDSQSILYLQMNNLKTEDTAMYYC |

TABLE 8
VH-Framework Region 4 (FR4):

| SEQ. ID. NO. | ADI Name | FR4 Sequence |
|---|---|---|
| 59 | ADI-40306 | WGQGTTVTVSS |
| | ADI-46663 | |
| | ADI-35281 | |
| 60 | ADI-40327 | WGQGTLVTVSS |
| 61 | ADI-40352 | WGQGTMVTVSS |
| | ADI-40360 | |

Light chain variable domain sequences of exemplary identified antibodies are presented below in Table 9.

TABLE 9
Variable Light Chain Amino Acid Sequence

| SEQ. | | |
|---|---|---|
| ID. | ADI | |
| NO. | Name | VL Protein Sequence |
| 44 | ADI- | DIQMTQSPSSLSASVGDRVTITCRASQSINSYLNWYQQKPGKA |
| | 40306 | PKLLIYAASSLQSGVPSRFSGSGSGTDFTLTISSLQPEDFATYYC |
| | | QESYSTPITFGGGTKVEIK |
| 45 | ADI- | DIQMTQSPSSLSASVGDRVTITCRASQSISSFLNWYQQKPGKAP |
| | 40327 | KLLISAASSLQSGVPSRFSGSGSGTDFTLTISSLQPEDFATYYCQ |
| | | QGHSTPPTFGGGTKVEIK |
| 46 | ADI- | DIQMTQSPSSLSASVGDRVTITCRASQSISSYLNWYQQKPGKAP |
| | 40352 | KLLIYAASSLQSGVPSRFSGSGSGTDFTLTISSLQPEDFATYYCQ |
| | | QSHNLPTFGGGTKVEIK |
| 47 | ADI- | DIQMTQSPSSLSASVGDRVTITCRASQSISSYLNWYQQKPGKAP |
| | 40360 | KLLIYAASSLQSGVPSRFSGSGSGTDFTLTISSLQPEDFATYYCQ |
| | | QSHNLPTFGGGTKVEIK |
| 48 | ADI- | DIVMTQSPLSLPVTPGEPASISCRSSKSLLHSNGNTYLYWYLQK |
| | 46663 | PGQSPQLLIYRMSNLASGVPDRFSGSGSGTDFTLKISRVEAEDV |
| | | GVYYCMQHLEYPFTFGGGTKVEIK |
| 49 | ADI- | DIQLTQSPPSVPVTPGESVSISCRSSKSLLHSNGNTYLYWFLQRP |
| | 35281 | GQSPQLLIYRKSNLASGVPDRFSGSGSGTAFTLRISRVEAEDVG |
| | | VYYCMQHLEYPFTFGGGTKLEIK |

The present disclosure describes FR and CDR sequences within these exemplified LC variable domains as set forth below in Tables 10-17.

TABLE 10
VL-CDR1:

| SEQ. ID. NO. | ADI Name | VL CDR1 Sequence |
|---|---|---|
| 15 | ADI-40306 | RASQSINSYLN |
| 16 | ADI-40327 | RASQSISSFLN |
| 17 | ADI-40352 | RASQSISSYLN |
| | ADI-40360 | |
| 18 | ADI-46663 | RSSKSLLHSNGNTYLY |
| | ADI-35281 | |

TABLE 11
VL-CDR2:

| SEQ. ID. NO. | ADI Name | VL CDR2 Sequence |
|---|---|---|
| 19 | ADI-40306 | AASSLQS |
| | ADI-40327 | |
| | ADI-40352 | |
| | ADI-40360 | |
| 20 | ADI-46663 | RMSNLAS |
| 21 | ADI-35281 | RKSNLAS |

TABLE 12
VL CDR3:

| SEQ. ID. NO. | ADI Name | VL CDR3 Sequence |
|---|---|---|
| 22 | ADI-40306 | QESYSTPIT |
| 23 | ADI-40327 | QQGHSTPPT |
| 24 | ADI-40352 | QQSHNLPT |
| | ADI-40360 | |
| 25 | ADI-46663 | MQHLEYPFT |
| | ADI-35281 | |

TABLE 13
VL FR1:

| SEQ. ID. NO. | ADI Name | VL FR1 Sequence |
|---|---|---|
| 62 | ADI-40306 | DIQMTQSPSSLSASVGDRVTITC |
| | ADI-40327 | |
| | ADI-40352 | |
| | ADI-40360 | |
| 63 | ADI-46663 | DIVMTQSPLSLPVTPGEPASISC |
| 64 | ADI-35281 | DIQLTQSPPSVPVTPGESVSISC |

TABLE 14
VL FR2:

| SEQ. ID. NO. | ADI Name | VL FR2 Sequence |
|---|---|---|
| 68 | ADI-40306 | WYQQKPGKAPKLLIY |
| | ADI-40352 | |
| | ADI-40360 | |
| 69 | ADI-40327 | WYQQKPGKAPKLLIS |
| 70 | ADI-46663 | WYLQKPGQSPQLLIY |
| 71 | ADI-35281 | WFLQRPGQSPQLLIY |

TABLE 15
VL FR3:

| SEQ. ID. NO. | ADI Name | VL FR3 Sequence |
|---|---|---|
| 74 | ADI-40306 | GVPSRFSGSGSGTDFTLTISSLQPEDFATYYC |
| | ADI-40327 | |
| | ADI-40352 | |
| | ADI-40360 | |
| 75 | ADI-46663 | GVPDRFSGSGSGTDFTLKISRVEAEDVGVYYC |
| 76 | ADI-35281 | GVPDRFSGSGSGTAFTLRISRVEAEDVGVYYC |

TABLE 16
VL FR4:

| SEQ. ID. NO. | ADI Name | VL FR4 Sequence |
|---|---|---|
| 80 | ADI-40306 | FGGGTKVEIK |
| | ADI-40327 | |
| | ADI-40352 | |
| | ADI-40360 | |
| | ADI-46663 | |
| 81 | ADI-35281 | FGGGTKLEIK |

Additionally, nucleic acid sequences encoding the variable heavy and variable light chains of these exemplary antibodies were determined. See Tables 17 and 18.

TABLE 17
Variable Heavy Chain Nucleic Acid Sequence

| SEQ. | | |
|---|---|---|
| ID. | ADI | |
| NO. | Name | VH DNA Sequence |
| 26 | ADI- | CAGGTGCAGCTGGTGGAGTCTGGGGGAGGCGTGGTCCAGCC |
| | 40306 | TGGGAGGTCCCTGAGACTCTCCTGTGCAGCGTCTGGATTCA |
| | | CCTTCAGTAGCTATGGCATGCACTGGGTCCGCCAGGCTCCA |
| | | GGCAAGGGGCTGGAGTGGGTGGCAGTTATATCGTATGATGG |
| | | AAGTAATAAATACTATGCATTTTCCGTGAAGGGCCGATTCA |
| | | CCATCTCCAGAGACAATTCCAAGAACACGCTGTATCTGCAA |
| | | ATGAACAGCCTGAGAGTCGAGGACACGGCGGTGTACTACTG |
| | | CGCCAGGGTCAGACGCATAGCAGGCAGAGCAGGGTACGGT |
| | | ATGGACGTTTGGGGCCAGGGAACAACTGTCACCGTCTCCTC |
| | | A |
| 27 | ADI- | CAGGTGCAGCTGCAGGAGTCGGGCCCAGGACTGGTGAAGC |
| | 40327 | CTTCGGAGACCCTGTCCCTCACCTGCGCTGTCTCTGGTTACT |
| | | CCATCAGCAGTGGTTACTACTGGGGCTGGATCCGGCAGCCC |
| | | CCAGGGAAGGGGCTGGAGTGGATTGGGAGTATCTATCATAG |
| | | TGGGAACACCTACTACAGGCCGTCCCTCAAGAGTCGAGTCA |
| | | CCATATCAGTAGACACGTCCAAGAACCAGTTCTCCCTGAAG |
| | | CTGAGTTCTGTGACCGCCGCAGACACGGCGGTGTACTACTG |
| | | CGCCAGGGGTAAGGGCGGCAGCTGGACAGCATTCGGGCCA |
| | | TGGGGACAGGGTACATTGGTCACCGTCTCCTCA |
| 28 | ADI- | CAGCTGCAGCTGCAGGAGTCGGGCCCAGGACTGGTGAAGCC |
| | 40352 | TTCGGAGACCCTGTCCCTCACCTGCACTGTCTCTGGTGGCTC |
| | | CATCAGCAGTAGTAGTTACGCATGGGGCTGGATCCGCCAGC |
| | | CCCCAGGGAAGAGGCTGGAGTGGATTGGGAGTATCTATTAT |
| | | ACTGGGAGCACCTACTACAACCCGTCCCTCAAGAGTCGAGT |
| | | CACCATATCCGTAGACACGTCCAAGAACCAGTTCTCCCTGA |
| | | AGCTGAGTTCTGTGACCGCCGCAGACACGGCGGTGTACTAC |
| | | TGCTTGAGAGGCCACAGAAGAGACTATATAGCATTCGACAT |
| | | ATGGGGTCAGGGTACAATGGTCACCGTCTCCTCA |
| 29 | ADI- | CAGCTGCAGCTGCAGGAGTCGGGCCCAGGACTGGTGAAGCC |
| | 40360 | TTCGGAGACCCTGTCCCTCACCTGCACTGTCTCTGGTGGCTC |
| | | CATCAGCAGTAGTAGTTACGCATGGGGCTGGATCCGCCAGC |
| | | CCCCAGGGAAGAGGCTGGAGTGGATTGGGAGTATCTATTAT |
| | | ACTGGGAGCACCTACTACAACCCGTCCCTCAAGAGTCGAGT |
| | | CACCATATCCGTAGACACGTCCAAGAACCAGTTCTCCCTGA |
| | | AGCTGAGTTCTGTGACCGCCGCAGACACGGCGGTGTACTAC |
| | | TGCGTGAGAGGCCACAGAAGAGACTATATAGCATTCGACAT |
| | | ATGGGGTCAGGGTACAATGGTCACCGTCTCCTCA |
| 30 | ADI- | GAGGTGCAGCTGGTGGAGTCTGGGGGAGGCTTGGTCCAGCC |
| | 46663 | TGGAGGGTCCCTGAGACTCTCCTGTGCAGCCTCTGGATTCAC |
| | | CTTCAATGCCTACGCCATGAACTGGGTCCGCCAGGCTCCAG |
| | | GGAAGGGGCTGGAGTGGGTTGCTCGTATAAGAAGTAAAAG |
| | | TAACAATTACGCAACATATTACGCCGATTCTGTGAAAGACA |
| | | GATTCACCATCTCAAGAGATGATTCAAAGAACTCACTGTAT |
| | | CTGCAAATGAACAGCCTGAAAACCGAGGACACGGCCGTGT |
| | | ATTACTGTGTGAGACAGTCCTATGGTAACTCCAACTATGCTA |
| | | TGGACTACTGGGGGCAAGGGACCACGGTCACCGTCTCCTCA |
| 31 | ADI- | GAGGTGAAGCTGGAGCAGTCTGGTGGAGGATTGGTGCAGCC |
| | 35281 | TAAAGGGTCATTGAAACTCTCATGTGTAGCCTCTGGATTCAC |
| | | CTTCAATGCCTACGCCATGAACTGGGTCCGCCAGGCTCCAG |
| | | GACAGGGTTTGGAATGGGTTGCTCGCATAAGAAGTAAAAGT |
| | | AATAATTATGCAACATATTATGCCGCTTCAGTGAAAGACAG |
| | | GTTCACCATCTCCAGAGATGATTCACAAAGCATACTCTATCT |
| | | GCAAATGAACAACTTGAAAACTGAGGACACAGCCATGTATT |
| | | ACTGTGTGAGACAGTCCTATGGTAACTCCAACTATGCTATG |
| | | GACTACTGGGGCCAAGGCACCACGGTCACCGTCTCCTCA |

TABLE 18
Variable Light Chain Nucleic Acid Sequence

| SEQ. | | |
|---|---|---|
| ID. | ADI | |
| NO. | Name | VL DNA Sequence |
| 32 | ADI- | GACATCCAGATGACCCAGTCTCCATCCTCCCTGTCTGCATCT |
| | 40306 | GTAGGAGACAGAGTCACCATCACTTGCCGGGCAAGTCAGAG |
| | | CATTAGCAGCTATTTAAATTGGTATCAGCAGAAACCAGGGA |
| | | AAGCCCCTAAGCTCCTGATCTATGCTGCATCCAGTTTGCAAA |
| | | GTGGGGTCCCATCAAGGTTCAGTGGCAGTGGATCTGGGACA |
| | | GATTTCACTCTCACCATCAGCAGTCTGCAACCTGAAGATTTT |
| | | GCAACTTACTACTGTCAGCAAGCATTCAGTCACCCTCCTACT |
| | | TTTGGCGGAGGGACCAAGGTTGAGATCAAA |
| 33 | ADI- | GACATCCAGATGACCCAGTCTCCATCCTCCCTGTCTGCATCT |
| | 40327 | GTAGGAGACAGAGTCACCATCACTTGCCGGGCAAGTCAGAG |
| | | CATTAGCAGCTTTTTAAATTGGTATCAGCAGAAACCAGGGA |
| | | AAGCCCCTAAGCTCCTGATCTCTGCTGCATCCAGTTTGCAAA |
| | | GTGGGGTCCCATCAAGGTTCAGTGGCAGTGGATCTGGGACA |
| | | GATTTCACTCTCACCATCAGCAGTCTGCAACCTGAAGATTTT |
| | | GCAACTTACTACTGTCAGCAAGGACACTCCACTCCTCCTACT |
| | | TTTGGCGGAGGGACCAAGGTTGAGATCAAA |
| 34 | ADI- | GACATCCAGATGACCCAGTCTCCATCCTCCCTGTCTGCATCT |
| | 40352 | GTAGGAGACAGAGTCACCATCACTTGCCGGGCAAGTCAGAG |
| | | CATTAGCAGCTATTTAAATTGGTATCAGCAGAAACCAGGGA |
| | | AAGCCCCTAAGCTCCTGATCTATGCTGCATCCAGTTTGCAAA |
| | | GTGGGGTCCCATCAAGGTTCAGTGGCAGTGGATCTGGGACA |
| | | GATTTCACTCTCACCATCAGCAGTCTGCAACCTGAAGATTTT |
| | | GCAACTTACTACTGTCAGCAAAGCCACAACCTCCCTACTTTT |
| | | GGCGGAGGGACCAAGGTTGAGATCAAA |
| 35 | ADI- | GACATCCAGATGACCCAGTCTCCATCCTCCCTGTCTGCATCT |
| | 40360 | GTAGGAGACAGAGTCACCATCACTTGCCGGGCAAGTCAGAG |
| | | CATTAGCAGCTATTTAAATTGGTATCAGCAGAAACCAGGGA |
| | | AAGCCCCTAAGCTCCTGATCTATGCTGCATCCAGTTTGCAAA |
| | | GTGGGGTCCCATCAAGGTTCAGTGGCAGTGGATCTGGGACA |
| | | GATTTCACTCTCACCATCAGCAGTCTGCAACCTGAAGATTTT |
| | | GCAACTTACTACTGTCAGCAAAGCCACAACCTCCCTACTTTT |
| | | GGCGGAGGGACCAAGGTTGAGATCAAA |
| 36 | ADI- | GATATTGTGATGACTCAGTCTCCACTCTCCCTGCCCGTCACC |
| | 46663 | CCTGGAGAGCCGGCCTCCATCTCCTGCAGGTCTAGTAAGAG |
| | | CCTCCTGCATAGTAATGGAAACACTTATTTGTATTGGTACCT |
| | | GCAGAAGCCAGGGCAGTCTCCACAGCTCCTGATCTATCGGA |
| | | TGTCTAATCTTGCCTCCGGGGTCCCTGACAGGTTCAGTGGCA |
| | | GTGGATCAGGCACAGATTTTACACTGAAAATCAGCAGAGTG |
| | | GAGGCTGAGGATGTTGGGGTTTATTACTGCATGCAACATCT |
| | | AGAATATCCTTTCACGTTCGGCGGAGGGACCAAGGTGGAGA |
| | | TCAAA |
| 37 | ADI- | GACATCCAGCTCACTCAGTCTCCACCCTCTGTACCTGTCACT |
| | 35281 | CCTGGAGAGTCAGTATCCATCTCCTGCAGGTCTAGTAAGAG |
| | | TCTCCTGCATAGTAATGGCAACACTTACTTGTATTGGTTCCT |
| | | GCAGAGGCCAGGCCAGTCTCCTCAGCTCCTGATATATCGGA |
| | | AGTCCAACCTTGCCTCAGGAGTCCCAGACAGGTTCAGTGGC |
| | | AGTGGGTCAGGAACTGCTTTCACACTGAGAATCAGTAGAGT |
| | | GGAGGCTGAGGATGTGGGTGTTTATTACTGTATGCAACATC |
| | | TAGAATATCCTTTCACGTTCGGAGGGGGGACCAAGTTGGAA |
| | | ATCAAA |

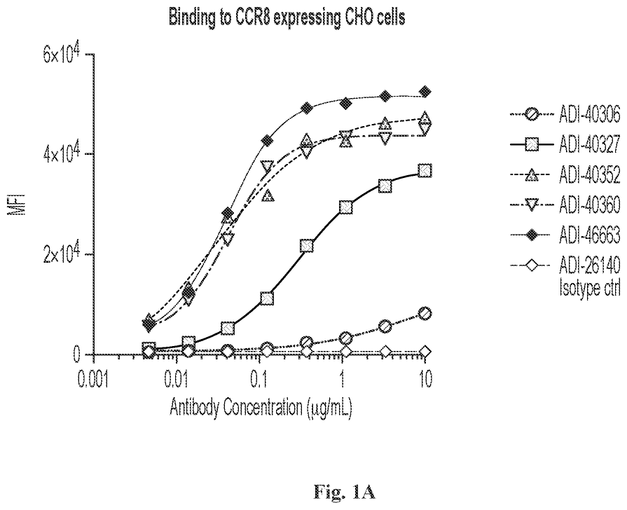

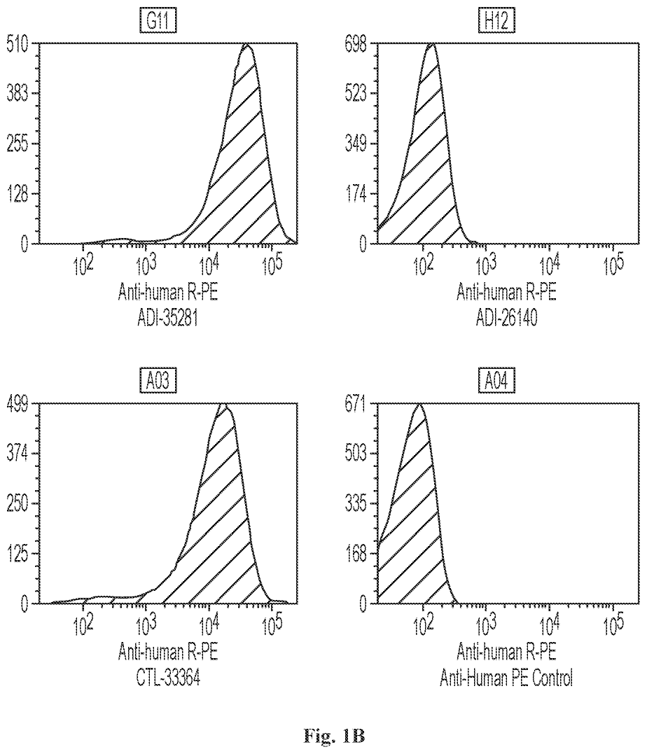

Certain binding properties and biophysical characteristics of each of the discovered antibodies was evaluated. As demonstrated in FIG. 1A-1B and Table 19, each of the discovered antibodies bound to human CCR8 expressed on mammalian cells. Additionally, each of the discovered antibodies was found to also bind cynomolgus monkey CCR8 expressed on mammalian cells.

TABLE 19
Binding of ADI-35281 to Human CCR8

| Antibody | MFI Fold over Background |
|---|---|
| ADI-35281 | 336 |
| ADI-26140 | 2 |
| CTL-33364 | 268 |

Binding to polyspecificity reagent; hydrophobic interaction chromatography retention time; Affinity-Capture Self-Interaction Nanoparticle Spectroscopy (AC-SINS) observed for each of these exemplary discovered antibodies was evaluated. Results of representative such tests are presented in Table 20.

TABLE 20
Biophysical Characteristics of Certain Discovered Antibodies

| | PSR | HIC | AC-SINS |
|---|---|---|---|
| Antibody | (Score 0-1) | (min) | Δλmax (nm) |
| ADI-40306 | 0.22 | 8.5 | 15.0 |
| ADI-40327 | 0.21 | 8.9 | 1.3 |
| ADI-40352 | 0.14 | 9.0 | 3.1 |
| ADI-40360 | 0.23 | 9.1 | 6.4 |
| ADI-46663 | 0.24 | 8.7 | 0.4 |
| ADI-35281 | 0.83 | 8.7 | N.D. |

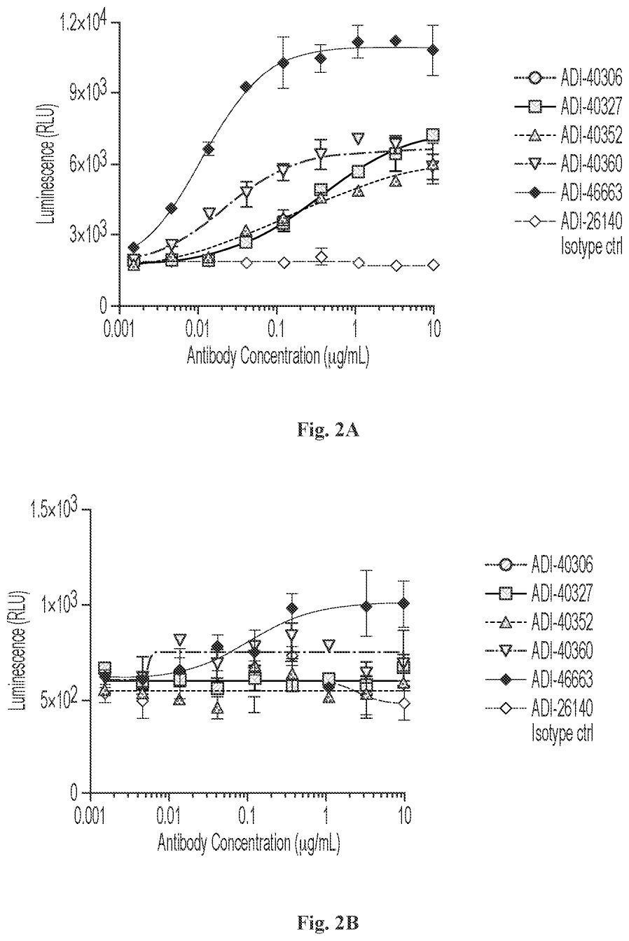

These discovered antibodies were tested for their ability to induce ADCC. FIGS. 2A and 2B show the induction of ADCC observed when the indicated antibody interacts with a high affinity (2A) and low affinity (2B) FcγRIIIa receptors.

These discovered antibodies were tested for their ability to compete for CCL1 binding to CCR8 and thus antagonize CCR8 signaling by the agonist CCL1. Table 21 demonstrates that the tested antibodies antagonized hCCL1-induced activation of Gαz and Gα15/16 signaling downstream of hCCR8.

TABLE 21
Antagonism of CCR8 Signaling

| | IC50 antagonism of EC80 | IC50 antagonism of EC80 |
|---|---|---|
| | Human CCL1 Gα15/16 | Human CCL1 Gαz |
| Antibody | Biosensor (M) | Biosensor (M) |
| ADI-40306 | 2.98E−08 | 1.89E−08 |
| ADI-40327 | 4.61E−09 | 5.46E−09 |
| ADI-40352 | 4.39E−10 | 2.39E−09 |
| ADI-40360 | 1.78E−09 | 1.14E−09 |
| ADI-46663 | 1.26E−09 | 1.79E−09 |
| ADI-35281 | N.D. | N.D. |
| CTL-33364 | 1.62E−09 | 7.98E−10 |
| MC148R | 3.35E−08 | 1.38E−08 |

CTL-33364 = Biolegend human CCR8 control IgG clone L263G8; MC148R = virokine from Molluscum contagiosum virus subtype 2 (MOCV).

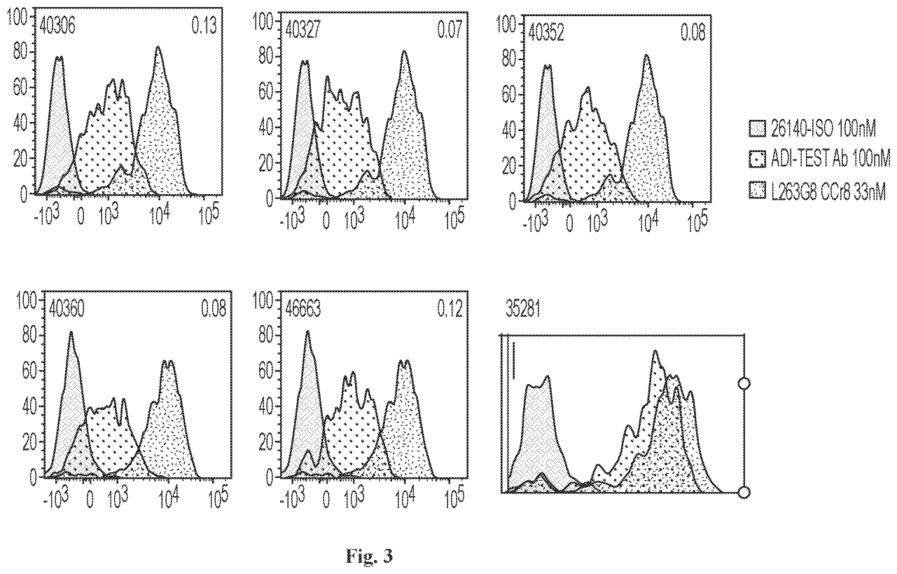

These discovered antibodies were tested for their ability to bind tumor infiltrating regulatory T cells. FIG. 3 demonstrates that each discovered antibody is able to bind to regulatory T-cells found in human tumors.

Those, skilled in the art, reading the present disclosure will appreciate that each of the exemplified antibodies, and antibody agents that are or include their antigen-binding elements, is useful to bind CCR8, and therefore in other contexts (e.g., in therapeutic and/or diagnostic contexts such as treatment of cancer (e.g., breast, colon, gastric, lung, and/or ovarian malignancy, etc), detection of CCR8, e.g., on cells, etc).

Furthermore, those skilled in the art will appreciate that components of these exemplified antibodies (e.g., antigen-binding fragments thereof and/or individual HC and/or LC variable domains, and/or FR and/or CDR sequences thereof, etc) may be useful, for example, in the development, production, and/or use of additional anti-CCR8 agents of interest. For example, heavy and light chains from different exemplary antibodies may, in some embodiments, be swapped to develop new antibodies that may be characterized for anti-CCR8 activity (ies), e.g., as described herein. Alternatively or additionally, individual HC and/or LC variable domains may be combined with other HC or LC variable domains in antibody agents whose anti-CCR8 activity may be assessed as described. Still further alternatively or additionally, individual FR and/or CDR sequence elements, and/or combinations thereof, may be used together with other FR and/or CDR sequences (e.g., from other exemplified antibodies and/or from different antibodies) in the development, production, and/or use of additional anti-CCR8 agents of interest and described herein.

### Example 3: Anti-CCR8 Agents Internalization

The present Example demonstrates a method for assessing internalization of anti-CCR8 agents described herein. Briefly, to determine internalization, Chinese Hamster Ovary Suspension (CHO-S) cells expressing hCCR8 and CHO-S cells expressing empty vector were collected from culture and cell density was assessed. Cells were diluted in cell culture media and plated (e.g., at 30,000 cells per well) in a 384 well plate. Anti-CCR8 antibodies were labeled with pH-sensitive dye conjugate Zenon pHrodo iFL IgG (Invitrogen Z25611) that is non-fluorescent outside of a cell, but fluorescent when inside acidic environments (e.g., when internalized). Labeling was achieved by mixing antibody with dye conjugate at room temperature for 15 minutes. 10 μL of labeled-antibody mix was transferred to the CHO-S cells such that, when added to cells, antibody mix was diluted to a final concentration of 20 μg/mL each of anti-CCR8 antibody and dye conjugate (e.g., 133 nM of anti-CCR8 antibody, 400 nM dye conjugate, 1:3 ratio). Cells were incubated at 37° C. for 6 hours with the labeled anti-CCR8 antibodies. Following incubation, 60 μL cold FACS buffer (2% FBS in PBS) was added to all wells to stop internalization. Next, cells were centrifuged and supernatant was removed. The pelleted cells were re-suspended in 10 μL of cold FACS buffer and mean fluorescence intensity (MFI) was measured on a flow cytometry instrument (excitation 505 nm, emission 530 nm).

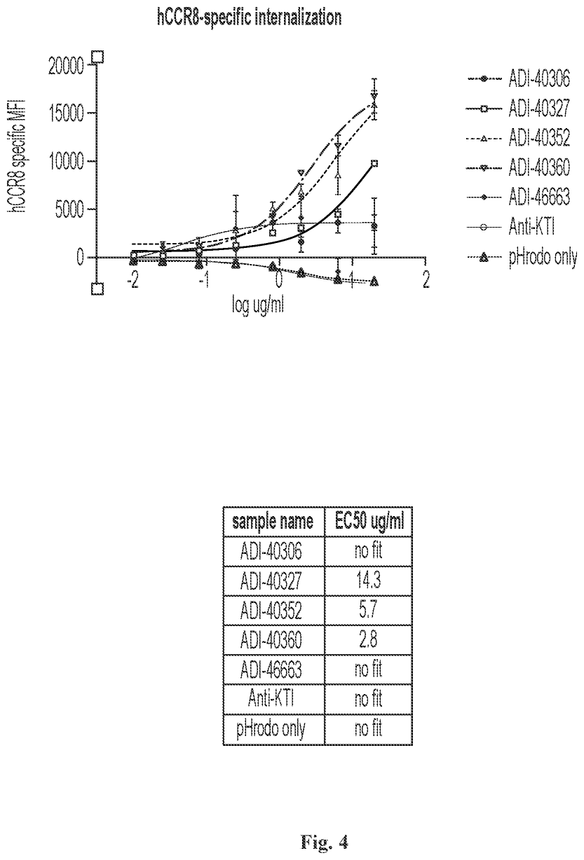

Antibody concentrations (log μg/mL) were plotted as a function of hCCR8 specific MFI. EC50 of hCCR8-specific internalization was calculated (FIG. 4). EC50 values for ADI-40327, ADI-40352, and ADI-40360 were determined as 14.3 μg/mL, 5.7 μg/mL, and 2.8 μg/mL, respectively.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. An antibody agent that is or comprises an antibody or antigen binding fragment thereof, which comprises a heavy chain variable domain and a light chain variable domain,
wherein the heavy chain variable domain has an amino acid sequence as set forth in one of:
a)
Xn1 FTFSSYGMH Xn2 VISYDGSNKYY AFSVKG Xn3 ARVRRIAGRAGYGMDV Xn4
b)
Xn1 YSISSGYYWG Xn2 SIYHSGNTYYRPSLKS Xn3 ARGKGGSWTAFGP Xn4
c)
Xn1 GSISSSSY AWG Xn2 SIYYTGSTYYNPSLKS Xn3 LRGHRRDYIAFDI Xn4
d)
Xn1 GSISSSSY AWG Xn2 SIYYTGSTYYNPSLKS Xn3 VRGHRRDYIAFDI Xn4
e)
Xn1 FTFNAYAMN Xn2 RIRSKSNNY ATYYADSVKD Xn3 VRQSYGNSNY
AMDY Xn4
f)
Xn1 FTFNAY AMN Xn2 RIRSKSNNY A TYY AASVKD Xn3 VRQSYGNSNY
AMDY Xn4;
and
wherein the light chain variable domain has an amino acid sequence as set forth in one of:
a)
Xn1 RASQSINSYLN Xn2 AASSLQS Xn3 QESYSTPIT Xn4
b)
Xn1 RASQSISSFLN Xn2 AASSLQS Xn3 QQGHSTPPT Xn4
c)
Xn1 RASQSISSYLN Xn2 AASSLQS Xn3 QQSHNLPT Xn4
d)
Xn1 RASQSISSYLN Xn2 AASSLQS Xn3 QQSHNLPT Xn4
e)
Xn1 RSSKSLLHSNGNTYL Y Xn2 RMSNLAS Xn3 MQHLEYPFT
Xn4
f)
Xn1 RSSKSLLHSNGNTYL Y Xn2 RKSNLAS Xn3 MQHLEYPFT
Xn4;
wherein each X, independently, is an amino acid, and each of n1; n2; n3; and n4, independently, is O to about 40.

2. An antibody agent that is or comprises an antibody or antigen binding fragment thereof, which antibody agent comprises a heavy chain variable domain and a light chain variable domain, wherein the heavy chain variable domain has an amino acid sequence including each of:
a sequence selected from SEQ ID Nos. 6-9;
a sequence selected from SEQ ID Nos. 10-14;
a sequence selected from SEQ ID Nos. 1-5; and
wherein the light chain variable domain has an amino acid sequence including each of:
a sequence selected from SEQ ID Nos. 15-18;
a sequence selected from SEQ ID Nos. 19-21;
and a sequence selected from SEQ ID Nos. 22-25.

3.-11. (canceled)

12. An antibody agent whose heavy chain variable domain amino acid sequence comprises or consists of a sequence substantially identical to a sequence selected from SEQ ID Nos. 38-43 and whose light chain variable domain amino acid sequence comprises or consists of a sequence substantially identical to a sequence selected from SEQ ID Nos. 44-49.

13. (canceled)

14. The antibody agent of claim 1, that is or comprises a monoclonal antibody, a single domain antibody, a single chain antibody, a Fab fragment, a F(ab′)2 fragment, a single chain variable fragment (scFv), a scFv-Fc fragment, a single chain antibody (scAb), or a single domain antibody.

15. The antibody agent of claim 1, that is or comprises a chimeric, humanized or fully human antibody or antigen binding fragment thereof.

16. The antibody agent of claim 1, that is or comprises an IgG 1, IgG2, IgG3, or IgG4 isotype antibody or antigen binding fragment thereof.

17. The antibody agent of claim 1, characterized in that it targets the extracellular domain of human CCR8.

18. The antibody agent of claim 17, characterized in that it binds cells expressing CCR8 on their surface.

19. The antibody agent of claim 18, wherein the cells are regulatory T cells (Tregs).

20. The antibody agent of claim 19, wherein the cells are tumor infiltrating Tregs.

21. (canceled)

22. A pharmaceutical composition that comprises or delivers an antibody agent of claim 1.

23. The pharmaceutical composition of claim 22, comprising the antibody agent.

24. (canceled)

25. (canceled)

26. The pharmaceutical composition of claim 22, which is a liquid composition.

27.-29. (canceled)

30. A cell expressing an antibody agent of claim 1.

31. (canceled)

32. The cell of claim 30, wherein the cell comprises a nucleic acid encoding the antibody agent of claim 1.

33. A method of treating a subject suffering from cancer, the method comprising a pharmaceutical composition of claim 22.

34. A method of producing an antibody agent, the method comprising culturing in vitro the cell of claim 30.

35.-37. (canceled)

38. A method of characterizing an antibody agent, the method comprising:
a) providing an antibody agent to be characterized that comprises an antibody whose heavy chain variable domain amino acid comprises or consists of a sequence substantially identical to one SEQ ID Nos. 38-43 and/or whose light chain variable domain sequence comprises or consists of a sequence substantially identical to one of SEQ ID Nos. 44-49, or an antigen-binding element thereof;
b) determining for the antibody agent one or more of:
i. human CCR8 binding
ii. cynomolgus monkey CCR8 binding
iii. antagonism of CCLI signaling
iv. antagonism of MCV148 signaling
v. induction of ADCC,
relative to that observed for a reference antibody whose heavy chain variable domain amino acid sequence is selected from the group consisting of SED ID Nos 38-43 and whose light chain variable domain amino acid sequence is selected from the group consisting of SEQ ID Nos 44-49, so that the provided antibody agent is characterized.

39. (canceled)

40. (canceled)

41. A method of manufacturing the pharmaceutical composition of claim 22 by formulating the antibody agent, or nucleic acid or cells, with at least one pharmaceutically acceptable carrier or excipient.

42. A method of detecting CCR8 by contacting a sample with an antibody agent of claim 1.

43. (canceled)

44. (canceled)


SEQUENCE LISTING (97 sequences)

SEQ ID NO: 1 - protein, 16 aa, Artificial Sequence
  Description of Artificial Sequence: Synthetic peptide
ARVRRIAGRAGYGMDV

SEQ ID NO: 2 - protein, 13 aa, Artificial Sequence
  Description of Artificial Sequence: Synthetic peptide
ARGKGGSWTAFGP

SEQ ID NO: 3 - protein, 13 aa, Artificial Sequence
  Description of Artificial Sequence: Synthetic peptide
LRGHRRDYIAFDI

SEQ ID NO: 4 - protein, 13 aa, Artificial Sequence
  Description of Artificial Sequence: Synthetic peptide
VRGHRRDYIAFDI

SEQ ID NO: 5 - protein, 14 aa, Artificial Sequence
  Description of Artificial Sequence: Synthetic peptide
VRQSYGNSNYAMDY

SEQ ID NO: 6 - protein, 9 aa, Artificial Sequence
  Description of Artificial Sequence: Synthetic peptide
FTFSSYGMH

SEQ ID NO: 7 - protein, 10 aa, Artificial Sequence
  Description of Artificial Sequence: Synthetic peptide
YSISSGYYWG

SEQ ID NO: 8 - protein, 11 aa, Artificial Sequence
  Description of Artificial Sequence: Synthetic peptide
GSISSSSYAWG

SEQ ID NO: 9 - protein, 9 aa, Artificial Sequence
  Description of Artificial Sequence: Synthetic peptide
FTFNAYAMN

SEQ ID NO: 10 - protein, 17 aa, Artificial Sequence
  Description of Artificial Sequence: Synthetic peptide
VISYDGSNKYYAFSVKG

SEQ ID NO: 11 - protein, 16 aa, Artificial Sequence
  Description of Artificial Sequence: Synthetic peptide
SIYHSGNTYYRPSLKS

SEQ ID NO: 12 - protein, 16 aa, Artificial Sequence
  Description of Artificial Sequence: Synthetic peptide
SIYYTGSTYYNPSLKS

SEQ ID NO: 13 - protein, 19 aa, Artificial Sequence
  Description of Artificial Sequence: Synthetic peptide
RIRSKSNNYATYYADSVKD

SEQ ID NO: 14 - protein, 19 aa, Artificial Sequence
  Description of Artificial Sequence: Synthetic peptide
RIRSKSNNYATYYAASVKD

SEQ ID NO: 15 - protein, 11 aa, Artificial Sequence
  Description of Artificial Sequence: Synthetic peptide
RASQSINSYLN

SEQ ID NO: 16 - protein, 11 aa, Artificial Sequence
  Description of Artificial Sequence: Synthetic peptide
RASQSISSFLN

SEQ ID NO: 17 - protein, 11 aa, Artificial Sequence
  Description of Artificial Sequence: Synthetic peptide
RASQSISSYLN

SEQ ID NO: 18 - protein, 16 aa, Artificial Sequence
  Description of Artificial Sequence: Synthetic peptide
RSSKSLLHSNGNTYLY

SEQ ID NO: 19 - protein, 7 aa, Artificial Sequence
  Description of Artificial Sequence: Synthetic peptide
AASSLQS

SEQ ID NO: 20 - protein, 7 aa, Artificial Sequence
  Description of Artificial Sequence: Synthetic peptide
RMSNLAS

SEQ ID NO: 21 - protein, 7 aa, Artificial Sequence
  Description of Artificial Sequence: Synthetic peptide
RKSNLAS

SEQ ID NO: 22 - protein, 9 aa, Artificial Sequence
  Description of Artificial Sequence: Synthetic peptide
QESYSTPIT

SEQ ID NO: 23 - protein, 9 aa, Artificial Sequence
  Description of Artificial Sequence: Synthetic peptide
QQGHSTPPT

SEQ ID NO: 24 - protein, 8 aa, Artificial Sequence
  Description of Artificial Sequence: Synthetic peptide
QQSHNLPT

SEQ ID NO: 25 - protein, 9 aa, Artificial Sequence
  Description of Artificial Sequence: Synthetic peptide
MQHLEYPFT

SEQ ID NO: 26 - DNA, 369 bp, Artificial Sequence
  Description of Artificial Sequence: Synthetic polynucleotide
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatcgtatgatggaagtaataaatactatgcattttccgtgaagggccgattcaccatctccagagacaattccaagaacacgctgtatctgcaaatgaacagcctgagagtcgaggacacggcggtgtactactgcgccagggtcagacgcatagcaggcagagcagggtacggtatggacgtttggggccagggaacaactgtcaccgtctcctca

SEQ ID NO: 27 - DNA, 360 bp, Artificial Sequence
  Description of Artificial Sequence: Synthetic polynucleotide
caggtgcagctgcaggagtcgggcccaggactggtgaagccttcggagaccctgtccctcacctgcgctgtctctggttactccatcagcagtggttactactggggctggatccggcagcccccagggaaggggctggagtggattgggagtatctatcatagtgggaacacctactacaggccgtccctcaagagtcgagtcaccatatcagtagacacgtccaagaaccagttctccctgaagctgagttctgtgaccgccgcagacacggcggtgtactactgcgccaggggtaagggcggcagctggacagcattcgggccatggggacagggtacattggtcaccgtctcctca

SEQ ID NO: 28 - DNA, 363 bp, Artificial Sequence
  Description of Artificial Sequence: Synthetic polynucleotide
cagctgcagctgcaggagtcgggcccaggactggtgaagccttcggagaccctgtccctcacctgcactgtctctggtggctccatcagcagtagtagttacgcatggggctggatccgccagcccccagggaagaggctggagtggattgggagtatctattatactgggagcacctactacaacccgtccctcaagagtcgagtcaccatatccgtagacacgtccaagaaccagttctccctgaagctgagttctgtgaccgccgcagacacggcggtgtactactgcttgagaggccacagaagagactatatagcattcgacatatggggtcagggtacaatggtcaccgtctcctca

SEQ ID NO: 29 - DNA, 363 bp, Artificial Sequence
  Description of Artificial Sequence: Synthetic polynucleotide
cagctgcagctgcaggagtcgggcccaggactggtgaagccttcggagaccctgtccctcacctgcactgtctctggtggctccatcagcagtagtagttacgcatggggctggatccgccagcccccagggaagaggctggagtggattgggagtatctattatactgggagcacctactacaacccgtccctcaagagtcgagtcaccatatccgtagacacgtccaagaaccagttctccctgaagctgagttctgtgaccgccgcagacacggcggtgtactactgcgtgagaggccacagaagagactatatagcattcgacatatggggtcagggtacaatggtcaccgtctcctca

SEQ ID NO: 30 - DNA, 369 bp, Artificial Sequence
  Description of Artificial Sequence: Synthetic polynucleotide
gaggtgcagctggtggagtctgggggaggcttggtccagcctggagggtccctgagactctcctgtgcagcctctggattcaccttcaatgcctacgccatgaactgggtccgccaggctccagggaaggggctggagtgggttgctcgtataagaagtaaaagtaacaattacgcaacatattacgccgattctgtgaaagacagattcaccatctcaagagatgattcaaagaactcactgtatctgcaaatgaacagcctgaaaaccgaggacacggccgtgtattactgtgtgagacagtcctatggtaactccaactatgctatggactactgggggcaagggaccacggtcaccgtctcctca

SEQ ID NO: 31 - DNA, 369 bp, Artificial Sequence
  Description of Artificial Sequence: Synthetic polynucleotide
gaggtgaagctggagcagtctggtggaggattggtgcagcctaaagggtcattgaaactctcatgtgtagcctctggattcaccttcaatgcctacgccatgaactgggtccgccaggctccaggacagggtttggaatgggttgctcgcataagaagtaaaagtaataattatgcaacatattatgccgcttcagtgaaagacaggttcaccatctccagagatgattcacaaagcatactctatctgcaaatgaacaacttgaaaactgaggacacagccatgtattactgtgtgagacagtcctatggtaactccaactatgctatggactactggggccaaggcaccacggtcaccgtctcctca

SEQ ID NO: 32 - DNA, 321 bp, Artificial Sequence
  Description of Artificial Sequence: Synthetic polynucleotide
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaagcattcagtcaccctcctacttttggcggagggaccaaggttgagatcaaa

SEQ ID NO: 33 - DNA, 321 bp, Artificial Sequence
  Description of Artificial Sequence: Synthetic polynucleotide
gacatccagatgacccagtctccatcctccctgtctgcatctgtaggagacagagtcaccatcacttgccgggcaagtcagagcattagcagctttttaaattggtatcagcagaaaccagggaaagcccctaagctcctgatctctgctgcatccagtttgcaaagtggggtcccatcaaggttcagtggcagtggatctgggacagatttcactctcaccatcagcagtctgcaacctgaagattttgcaacttactactgtcagcaaggacactccactcctcctacttttggcggagggaccaaggttgagatcaaa

SEQ ID NO: 34 - DNA, 318 bp, Artificial Sequence
  Description of Artificial Sequence: Synthetic polynucleotide
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaagccacaacctccctacttttggcggagggaccaaggttgagatcaaa

SEQ ID NO: 35 - DNA, 318 bp, Artificial Sequence; identical to SEQ ID NO: 34
  Description of Artificial Sequence: Synthetic polynucleotide

SEQ ID NO: 36 - DNA, 336 bp, Artificial Sequence
  Description of Artificial Sequence: Synthetic polynucleotide
gatattgtgatgactcagtctccactctccctgcccgtcacccctggagagccggcctccatctcctgcaggtctagtaagagcctcctgcatagtaatggaaacacttatttgtattggtacctgcagaagccagggcagtctccacagctcctgatctatcggatgtctaatcttgcctccggggtccctgacaggttcagtggcagtggatcaggcacagattttacactgaaaatcagcagagtggaggctgaggatgttggggtttattactgcatgcaacatctagaatatcctttcacgttcggcggagggaccaaggtggagatcaaa

SEQ ID NO: 37 - DNA, 336 bp, Artificial Sequence
  Description of Artificial Sequence: Synthetic polynucleotide
gacatccagctcactcagtctccaccctctgtacctgtcactcctggagagtcagtatccatctcctgcaggtctagtaagagtctcctgcatagtaatggcaacacttacttgtattggttcctgcagaggccaggccagtctcctcagctcctgatatatcggaagtccaaccttgcctcaggagtcccagacaggttcagtggcagtgggtcaggaactgctttcacactgagaatcagtagagtggaggctgaggatgtgggtgtttattactgtatgcaacatctagaatatcctttcacgttcggaggggggaccaagttggaaatcaaa

SEQ ID NO: 38 - protein, 123 aa, Artificial Sequence
  Description of Artificial Sequence: Synthetic polypeptide
QVQLVESGGGVVQPGRSLRLSCAASGFTFSSYGMHWVRQAPGKGLEWVAVISYDGSNKYYAFSVKGRFTISRDNSKNTLYLQMNSLRVEDTAVYYCARVRRIAGRAGYGMDVWGQGTTVTVSS

SEQ ID NO: 39 - protein, 120 aa, Artificial Sequence
  Description of Artificial Sequence: Synthetic polypeptide
QVQLQESGPGLVKPSETLSLTCAVSGYSISSGYYWGWIRQPPGKGLEWIGSIYHSGNTYYRPSLKSRVTISVDTSKNQFSLKLSSVTAADTAVYYCARGKGGSWTAFGPWGQGTLVTVSS

SEQ ID NO: 40 - protein, 121 aa, Artificial Sequence
  Description of Artificial Sequence: Synthetic polypeptide
QLQLQESGPGLVKPSETLSLTCTVSGGSISSSSYAWGWIRQPPGKRLEWIGSIYYTGSTYYNPSLKSRVTISVDTSKNQFSLKLSSVTAADTAVYYCLRGHRRDYIAFDIWGQGTMVTVSS

SEQ ID NO: 41 - protein, 121 aa, Artificial Sequence
  Description of Artificial Sequence: Synthetic polypeptide
QLQLQESGPGLVKPSETLSLTCTVSGGSISSSSYAWGWIRQPPGKRLEWIGSIYYTGSTYYNPSLKSRVTISVDTSKNQFSLKLSSVTAADTAVYYCVRGHRRDYIAFDIWGQGTMVTVSS

SEQ ID NO: 42 - protein, 123 aa, Artificial Sequence
  Description of Artificial Sequence: Synthetic polypeptide
EVQLVESGGGLVQPGGSLRLSCAASGFTFNAYAMNWVRQAPGKGLEWVARIRSKSNNYATYYADSVKDRFTISRDDSKNSLYLQMNSLKTEDTAVYYCVRQSYGNSNYAMDYWGQGTTVTVSS

SEQ ID NO: 43 - protein, 123 aa, Artificial Sequence
  Description of Artificial Sequence: Synthetic polypeptide
EVKLEQSGGGLVQPKGSLKLSCVASGFTFNAYAMNWVRQAPGQGLEWVARIRSKSNNYATYYAASVKDRFTISRDDSQSILYLQMNNLKTEDTAMYYCVRQSYGNSNYAMDYWGQGTTVTVSS

SEQ ID NO: 44 - protein, 107 aa, Artificial Sequence
  Description of Artificial Sequence: Synthetic polypeptide
DIQMTQSPSSLSASVGDRVTITCRASQSINSYLNWYQQKPGKAPKLLIYAASSLQSGVPSRFSGSGSGTDFTLTISSLQPEDFATYYCQESYSTPITFGGGTKVEIK

SEQ ID NO: 45 - protein, 107 aa, Artificial Sequence
  Description of Artificial Sequence: Synthetic polypeptide
DIQMTQSPSSLSASVGDRVTITCRASQSISSFLNWYQQKPGKAPKLLISAASSLQSGVPSRFSGSGSGTDFTLTISSLQPEDFATYYCQQGHSTPPTFGGGTKVEIK

SEQ ID NO: 46 - protein, 106 aa, Artificial Sequence
  Description of Artificial Sequence: Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSHNLPTFGGGTKVEIK

SEQ ID NO: 47 - protein, 106 aa, Artificial Sequence; identical to SEQ ID NO: 46
  Description of Artificial Sequence: Synthetic polypeptide

SEQ ID NO: 48 - protein, 112 aa, Artificial Sequence
  Description of Artificial Sequence: Synthetic polypeptide
DIVMTQSPLSLPVTPGEPASISCRSSKSLLHSNGNTYLYWYLQKPGQSPQLLIYRMSNLASGVPDRFSGSGSGTDFTLKISRVEAEDVGVYYCMQHLEYPFTFGGGTKVEIK

SEQ ID NO: 49 - protein, 112 aa, Artificial Sequence
  Description of Artificial Sequence: Synthetic polypeptide
DIQLTQSPPSVPVTPGESVSISCRSSKSLLHSNGNTYLYWFLQRPGQSPQLLIYRKSNLASGVPDRFSGSGSGTAFTLRISRVEAEDVGVYYCMQHLEYPFTFGGGTKLEIK

SEQ ID NO: 50 - protein, 26 aa, Artificial Sequence
  Description of Artificial Sequence: Synthetic peptide
QVQLVESGGGVVQPGRSLRLSCAASG

SEQ ID NO: 51 - protein, 26 aa, Artificial Sequence
  Description of Artificial Sequence: Synthetic peptide
QVQLQESGPGLVKPSETLSLTCAVSG

SEQ ID NO: 52 - protein, 26 aa, Artificial Sequence
  Description of Artificial Sequence: Synthetic peptide
QLQLQESGPGLVKPSETLSLTCTVSG

SEQ ID NO: 53 - protein, 26 aa, Artificial Sequence
  Description of Artificial Sequence: Synthetic peptide
EVQLVESGGGLVQPGGSLRLSCAASG

SEQ ID NO: 54 - protein, 26 aa, Artificial Sequence
  Description of Artificial Sequence: Synthetic peptide
EVKLEQSGGGLVQPKGSLKLSCVASG

SEQ ID NO: 55 - protein, 14 aa, Artificial Sequence
  Description of Artificial Sequence: Synthetic peptide
WVRQAPGKGLEWVA

SEQ ID NO: 56 - protein, 14 aa, Artificial Sequence
  Description of Artificial Sequence: Synthetic peptide
WIRQPPGKGLEWIG

SEQ ID NO: 57 - protein, 16 aa, Artificial Sequence
  Description of Artificial Sequence: Synthetic peptide
SIYYTGSTYYNPSLKS

SEQ ID NO: 58 - protein, 19 aa, Artificial Sequence
  Description of Artificial Sequence: Synthetic peptide
RIRSKSNNYATYYADSVKD

SEQ ID NO: 59 - protein, 14 aa, Artificial Sequence
  Description of Artificial Sequence: Synthetic peptide
WVRQAPGQGLEWVA

SEQ ID NO: 60 - protein, 11 aa, Artificial Sequence
  Description of Artificial Sequence: Synthetic peptide
WGQGTLVTVSS

SEQ ID NO: 61 - protein, 11 aa, Artificial Sequence
  Description of Artificial Sequence: Synthetic peptide
WGQGTMVTVSS

SEQ ID NO: 62 - protein, 23 aa, Artificial Sequence
  Description of Artificial Sequence: Synthetic peptide
DIQMTQSPSSLSASVGDRVTITC

SEQ ID NO: 63 - protein, 23 aa, Artificial Sequence
  Description of Artificial Sequence: Synthetic peptide
DIVMTQSPLSLPVTPGEPASISC

SEQ ID NO: 64 - protein, 23 aa, Artificial Sequence
  Description of Artificial Sequence: Synthetic peptide
DIQLTQSPPSVPVTPGESVSISC

SEQ ID NO: 65-67 - skipped (3 entries)

SEQ ID NO: 68 - protein, 15 aa, Artificial Sequence
  Description of Artificial Sequence: Synthetic peptide
WYQQKPGKAPKLLIY

SEQ ID NO: 69 - protein, 15 aa, Artificial Sequence
  Description of Artificial Sequence: Synthetic peptide
WYQQKPGKAPKLLIS

SEQ ID NO: 70 - protein, 15 aa, Artificial Sequence
  Description of Artificial Sequence: Synthetic peptide
WYLQKPGQSPQLLIY

SEQ ID NO: 71 - protein, 15 aa, Artificial Sequence
  Description of Artificial Sequence: Synthetic peptide
WFLQRPGQSPQLLIY

SEQ ID NO: 72-73 - skipped (2 entries)

SEQ ID NO: 74 - protein, 32 aa, Artificial Sequence
  Description of Artificial Sequence: Synthetic polypeptide
GVPSRFSGSGSGTDFTLTISSLQPEDFATYYC

SEQ ID NO: 75 - protein, 32 aa, Artificial Sequence
  Description of Artificial Sequence: Synthetic polypeptide
GVPDRFSGSGSGTDFTLKISRVEAEDVGVYYC

SEQ ID NO: 76 - protein, 32 aa, Artificial Sequence
  Description of Artificial Sequence: Synthetic polypeptide
GVPDRFSGSGSGTAFTLRISRVEAEDVGVYYC

SEQ ID NO: 77-79 - skipped (3 entries)

SEQ ID NO: 80 - protein, 10 aa, Artificial Sequence
  Description of Artificial Sequence: Synthetic peptide
FGGGTKVEIK

SEQ ID NO: 81 - protein, 10 aa, Artificial Sequence
  Description of Artificial Sequence: Synthetic peptide
FGGGTKLEIK

SEQ ID NO: 82 - protein, 30 aa, Artificial Sequence
  Description of Artificial Sequence: Synthetic polypeptide
RFTISRDNSKNTLYLQMNSLRVEDTAVYYC

SEQ ID NO: 83 - protein, 30 aa, Artificial Sequence
  Description of Artificial Sequence: Synthetic polypeptide
RVTISVDTSKNQFSLKLSSVTAADTAVYYC

SEQ ID NO: 84 - protein, 30 aa, Artificial Sequence
  Description of Artificial Sequence: Synthetic polypeptide
RFTISRDDSKNSLYLQMNSLKTEDTAVYYC

SEQ ID NO: 85 - protein, 30 aa, Artificial Sequence
  Description of Artificial Sequence: Synthetic polypeptide
RFTISRDDSQSILYLQMNNLKTEDTAMYYC

SEQ ID NO: 86 - protein, 11 aa, Artificial Sequence
  Description of Artificial Sequence: Synthetic peptide
WGQGTTVTVSS

SEQ ID NO: 87 - protein, 202 aa, Artificial Sequence
  Description of Artificial Sequence: Synthetic polypeptide
  MOD_RES (1)..(40): Any amino acid or absent
  SITE (1)..(40): This region may encompass 0-40 residues
  MOD_RES (50)..(89): Any amino acid or absent
  SITE (50)..(89): This region may encompass 0-40 residues
  MOD_RES (107)..(146): Any amino acid or absent
  SITE (107)..(146): This region may encompass 0-40 residues
  MOD_RES (163)..(202): Any amino acid or absent
  SITE (163)..(202): This region may encompass 0-40 residues
  See specification as filed for detailed description of substitutions and preferred embodiments
XXXXXXXXXXXXXXXXXXXXXXXXXXXXXXXXXXXXXXXXFTFSSYGMHXXXXXXXXXXXXXXXXXXXXXXXXXXXXXXXXXXXXXXXXVISYDGSNKYYAFSVKGXXXXXXXXXXXXXXXXXXXXXXXXXXXXXXXXXXXXXXXXARVRRIAGRAGYGMDVXXXXXXXXXXXXXXXXXXXXXXXXXXXXXXXXXXXXXXXX

SEQ ID NO: 88 - protein, 199 aa, Artificial Sequence
  Description of Artificial Sequence: Synthetic polypeptide
  MOD_RES (1)..(40): Any amino acid or absent
  SITE (1)..(40): This region may encompass 0-40 residues
  MOD_RES (51)..(90): Any amino acid or absent
  SITE (51)..(90): This region may encompass 0-40 residues
  MOD_RES (107)..(146): Any amino acid or absent
  SITE (107)..(146): This region may encompass 0-40 residues
  MOD_RES (160)..(199): Any amino acid or absent
  SITE (160)..(199): This region may encompass 0-40 residues
  See specification as filed for detailed description of substitutions and preferred embodiments
XXXXXXXXXXXXXXXXXXXXXXXXXXXXXXXXXXXXXXXXYSISSGYYWGXXXXXXXXXXXXXXXXXXXXXXXXXXXXXXXXXXXXXXXXSIYHSGNTYYRPSLKSXXXXXXXXXXXXXXXXXXXXXXXXXXXXXXXXXXXXXXXXARGKGGSWTAFGPXXXXXXXXXXXXXXXXXXXXXXXXXXXXXXXXXXXXXXXX

SEQ ID NO: 89 - protein, 200 aa, Artificial Sequence
  Description of Artificial Sequence: Synthetic polypeptide
  MOD_RES (1)..(40): Any amino acid or absent
  SITE (1)..(40): This region may encompass 0-40 residues
  MOD_RES (52)..(91): Any amino acid or absent
  SITE (52)..(91): This region may encompass 0-40 residues
  MOD_RES (108)..(147): Any amino acid or absent
  SITE (108)..(147): This region may encompass 0-40 residues
  MOD_RES (161)..(200): Any amino acid or absent
  SITE (161)..(200): This region may encompass 0-40 residues
  See specification as filed for detailed description of substitutions and preferred embodiments
XXXXXXXXXXXXXXXXXXXXXXXXXXXXXXXXXXXXXXXXGSISSSSYAWGXXXXXXXXXXXXXXXXXXXXXXXXXXXXXXXXXXXXXXXXSIYYTGSTYYNPSLKSXXXXXXXXXXXXXXXXXXXXXXXXXXXXXXXXXXXXXXXXLRGHRRDYIAFDIXXXXXXXXXXXXXXXXXXXXXXXXXXXXXXXXXXXXXXXX

SEQ ID NO: 90 - protein, 200 aa, Artificial Sequence
  Description of Artificial Sequence: Synthetic polypeptide
  MOD_RES (1)..(40): Any amino acid or absent
  SITE (1)..(40): This region may encompass 0-40 residues
  MOD_RES (52)..(91): Any amino acid or absent
  SITE (52)..(91): This region may encompass 0-40 residues
  MOD_RES (108)..(147): Any amino acid or absent
  SITE (108)..(147): This region may encompass 0-40 residues
  MOD_RES (161)..(200): Any amino acid or absent
  SITE (161)..(200): This region may encompass 0-40 residues
  See specification as filed for detailed description of substitutions and preferred embodiments
XXXXXXXXXXXXXXXXXXXXXXXXXXXXXXXXXXXXXXXXGSISSSSYAWGXXXXXXXXXXXXXXXXXXXXXXXXXXXXXXXXXXXXXXXXSIYYTGSTYYNPSLKSXXXXXXXXXXXXXXXXXXXXXXXXXXXXXXXXXXXXXXXXVRGHRRDYIAFDIXXXXXXXXXXXXXXXXXXXXXXXXXXXXXXXXXXXXXXXX

SEQ ID NO: 91 - protein, 202 aa, Artificial Sequence
  Description of Artificial Sequence: Synthetic polypeptide
  MOD_RES (1)..(40): Any amino acid or absent
  SITE (1)..(40): This region may encompass 0-40 residues
  MOD_RES (50)..(89): Any amino acid or absent
  SITE (50)..(89): This region may encompass 0-40 residues
  MOD_RES (109)..(148): Any amino acid or absent
  SITE (109)..(148): This region may encompass 0-40 residues
  MOD_RES (163)..(202): Any amino acid or absent
  SITE (163)..(202): This region may encompass 0-40 residues
  See specification as filed for detailed description of substitutions and preferred embodiments
XXXXXXXXXXXXXXXXXXXXXXXXXXXXXXXXXXXXXXXXFTFNAYAMNXXXXXXXXXXXXXXXXXXXXXXXXXXXXXXXXXXXXXXXXRIRSKSNNYATYYADSVKDXXXXXXXXXXXXXXXXXXXXXXXXXXXXXXXXXXXXXXXXVRQSYGNSNYAMDYXXXXXXXXXXXXXXXXXXXXXXXXXXXXXXXXXXXXXXXX

SEQ ID NO: 92 - protein, 202 aa, Artificial Sequence
  Description of Artificial Sequence: Synthetic polypeptide
  MOD_RES (1)..(40): Any amino acid or absent
  SITE (1)..(40): This region may encompass 0-40 residues
  MOD_RES (50)..(89): Any amino acid or absent
  SITE (50)..(89): This region may encompass 0-40 residues
  MOD_RES (109)..(148): Any amino acid or absent
  SITE (109)..(148): This region may encompass 0-40 residues
  MOD_RES (163)..(202): Any amino acid or absent
  SITE (163)..(202): This region may encompass 0-40 residues
  See specification as filed for detailed description of substitutions and preferred embodiments
XXXXXXXXXXXXXXXXXXXXXXXXXXXXXXXXXXXXXXXXFTFNAYAMNXXXXXXXXXXXXXXXXXXXXXXXXXXXXXXXXXXXXXXXXRIRSKSNNYATYYAASVKDXXXXXXXXXXXXXXXXXXXXXXXXXXXXXXXXXXXXXXXXVRQSYGNSNYAMDYXXXXXXXXXXXXXXXXXXXXXXXXXXXXXXXXXXXXXXXX

SEQ ID NO: 93 - protein, 187 aa, Artificial Sequence
  Description of Artificial Sequence: Synthetic polypeptide
  MOD_RES (1)..(40): Any amino acid or absent
  SITE (1)..(40): This region may encompass 0-40 residues
  MOD_RES (52)..(91): Any amino acid or absent
  SITE (52)..(91): This region may encompass 0-40 residues
  MOD_RES (99)..(138): Any amino acid or absent
  SITE (99)..(138): This region may encompass 0-40 residues
  MOD_RES (148)..(187): Any amino acid or absent
  SITE (148)..(187): This region may encompass 0-40 residues
  See specification as filed for detailed description of substitutions and preferred embodiments
XXXXXXXXXXXXXXXXXXXXXXXXXXXXXXXXXXXXXXXXRASQSINSYLNXXXXXXXXXXXXXXXXXXXXXXXXXXXXXXXXXXXXXXXXAASSLQSXXXXXXXXXXXXXXXXXXXXXXXXXXXXXXXXXXXXXXXXQESYSTPITXXXXXXXXXXXXXXXXXXXXXXXXXXXXXXXXXXXXXXXX

SEQ ID NO: 94 - protein, 187 aa, Artificial Sequence
  Description of Artificial Sequence: Synthetic polypeptide
  MOD_RES (1)..(40): Any amino acid or absent
  SITE (1)..(40): This region may encompass 0-40 residues
  MOD_RES (52)..(91): Any amino acid or absent
  SITE (52)..(91): This region may encompass 0-40 residues
  MOD_RES (99)..(138): Any amino acid or absent
  SITE (99)..(138): This region may encompass 0-40 residues
  MOD_RES (148)..(187): Any amino acid or absent
  SITE (148)..(187): This region may encompass 0-40 residues
  See specification as filed for detailed description of substitutions and preferred embodiments
XXXXXXXXXXXXXXXXXXXXXXXXXXXXXXXXXXXXXXXXRASQSISSFLNXXXXXXXXXXXXXXXXXXXXXXXXXXXXXXXXXXXXXXXXAASSLQSXXXXXXXXXXXXXXXXXXXXXXXXXXXXXXXXXXXXXXXXQQGHSTPPTXXXXXXXXXXXXXXXXXXXXXXXXXXXXXXXXXXXXXXXX

SEQ ID NO: 95 - protein, 186 aa, Artificial Sequence
  Description of Artificial Sequence: Synthetic polypeptide
  MOD_RES (1)..(40): Any amino acid or absent
  SITE (1)..(40): This region may encompass 0-40 residues
  MOD_RES (52)..(91): Any amino acid or absent
  SITE (52)..(91): This region may encompass 0-40 residues
  MOD_RES (99)..(138): Any amino acid or absent
  SITE (99)..(138): This region may encompass 0-40 residues
  MOD_RES (147)..(186): Any amino acid or absent
  SITE (147)..(186): This region may encompass 0-40 residues
  See specification as filed for detailed description of substitutions and preferred embodiments
XXXXXXXXXXXXXXXXXXXXXXXXXXXXXXXXXXXXXXXXRASQSISSYLNXXXXXXXXXXXXXXXXXXXXXXXXXXXXXXXXXXXXXXXXAASSLQSXXXXXXXXXXXXXXXXXXXXXXXXXXXXXXXXXXXXXXXXQQSHNLPTXXXXXXXXXXXXXXXXXXXXXXXXXXXXXXXXXXXXXXXX

SEQ ID NO: 96 - protein, 192 aa, Artificial Sequence
  Description of Artificial Sequence: Synthetic polypeptide
  MOD_RES (1)..(40): Any amino acid or absent
  SITE (1)..(40): This region may encompass 0-40 residues
  MOD_RES (57)..(96): Any amino acid or absent
  SITE (57)..(96): This region may encompass 0-40 residues
  MOD_RES (104)..(143): Any amino acid or absent
  SITE (104)..(143): This region may encompass 0-40 residues
  MOD_RES (153)..(192): Any amino acid or absent
  SITE (153)..(192): This region may encompass 0-40 residues
  See specification as filed for detailed description of substitutions and preferred embodiments
XXXXXXXXXXXXXXXXXXXXXXXXXXXXXXXXXXXXXXXXRSSKSLLHSNGNTYLYXXXXXXXXXXXXXXXXXXXXXXXXXXXXXXXXXXXXXXXXRMSNLASXXXXXXXXXXXXXXXXXXXXXXXXXXXXXXXXXXXXXXXXMQHLEYPFTXXXXXXXXXXXXXXXXXXXXXXXXXXXXXXXXXXXXXXXX

SEQ ID NO: 97 - protein, 192 aa, Artificial Sequence
  Description of Artificial Sequence: Synthetic polypeptide
  MOD_RES (1)..(40): Any amino acid or absent
  SITE (1)..(40): This region may encompass 0-40 residues
  MOD_RES (57)..(96): Any amino acid or absent
  SITE (57)..(96): This region may encompass 0-40 residues
  MOD_RES (104)..(143): Any amino acid or absent
  SITE (104)..(143): This region may encompass 0-40 residues
  MOD_RES (153)..(192): Any amino acid or absent
  SITE (153)..(192): This region may encompass 0-40 residues
  See specification as filed for detailed description of substitutions and preferred embodiments
XXXXXXXXXXXXXXXXXXXXXXXXXXXXXXXXXXXXXXXXRSSKSLLHSNGNTYLYXXXXXXXXXXXXXXXXXXXXXXXXXXXXXXXXXXXXXXXXRKSNLASXXXXXXXXXXXXXXXXXXXXXXXXXXXXXXXXXXXXXXXXMQHLEYPFTXXXXXXXXXXXXXXXXXXXXXXXXXXXXXXXXXXXXXXXX